(19) United States
(12) Patent Application Publication
(10) Pub. No.: US 2026/0209722 A1
(43) Pub. Date: Jul. 23, 2026

(54) MEANS AND METHODS TO TARGET ENDOGENOUS CONDENSATES

(71) Applicants: MAX-PLANCK-GESELLSCHAFT ZUR FÖRDERUNG DER WISSENSCHAFTEN E. V., München (DE); MASSACHUSETTS INSTITUTE OF TECHNOLOGY, Cambridge, MA (US)

(72) Inventors: Ibrahim CISSÉ, Freiburg (DE); Choongman LEE

(73) Assignees: MAX-PLANCK-GESELLSCHAFT ZUR FÖRDERUNG DER WISSENSCHAFTEN E. V., München (DE); MASSACHUSETTS INSTITUTE OF TECHNOLOGY, Cambridge, MA (US)

(21) Appl. No.: 19/139,976

(22) PCT Filed: Dec. 18, 2023
(86) PCT No.: PCT/EP2023/086260

§ 371 Date: Jun. 17, 2025

Related U.S. Application Data
(60) Provisional application No. 63/387,982, filed on Dec. 19, 2022.

(30) Foreign Application Priority Data
May 4, 2023 (EP) 23171660.6

(51) Int. Cl. C12N 9/12; C07K 14/47; C07K 16/14; C12N 15/85

(52) U.S. Cl. CPC C12N 9/1247; C07K 14/4702; C07K 16/14; C12N 15/85; C07K 2317/622; C07K 2319/60; C07K 2319/70; C12Y 207/07006

(57) ABSTRACT

The invention relates to a system for modifying a target biomolecule that is associated to an endogenous intracellular condensate formed by an intracellular protein comprising an Intrinsically Disordered Region (IDR), or to a system for modifying a chemical or physical property of an endogenous intracellular condensate. The system of the invention comprises as its minimal components an IDR polypeptide sequence tract comprising the Intrinsically Disordered Region of the intracellular protein, and an effector polypeptide domain capable of covalently modifying said target biomolecule.

This application claims the priority of U.S. provisional application 63/387,982, submitted 19 Dec. 2022, and of European application EP23171660.6, submitted 4 May 2023, both of which are incorporated herein in their entirety.

### FIELD

The present invention relates to multimodular polypeptide constructs facilitating the manipulation of endogenous intracellular condensates, such as those generated by liquid-liquid phase separation or biological phase transitions, of proteins containing intrinsically disordered regions (IDR). The invention further relates to methods using the multimodular polypeptides in analyzing the components of the condensates.

### BACKGROUND

The emerging science of liquid-liquid phase separation and biomolecular condensates impacts various fields, including cell biology, neuroscience, and cancer biology. In particular, transcriptional condensates are drawing attention because their role in transcription may be essential to understand gene expression in various cell types, especially stem cells and cancer cells. However, the available tools to study these phenomena are limited because condensates are membrane-less, liquid-like, and usually small (less than 1 μm). In this sense, microscopic methods, which can visualize the condensates without manipulating cells, are mainly used to study condensates. However, this is not sufficient to achieve genome-wide and proteome-wide information about the condensates' role. Therefore, new systems and tools are desirable to study liquid-like condensates while keeping the main features of the condensates intact during the experiment.

Liu et al. (FRONTIERS IN ONCOLOGY 12, 21 Mar. 2022) report on posttranslational modifications of BRD4.

Chiang (DRUG DISCOVERY TODAY: TECHNOLOGIES 19, (2016), 17-22) discusses phosphorylation of BRD4 and its interaction with drugs targeting BRD4.

Vershininz et al. (SCIENCE ADVANCES 22, 26 May 2021) report that methylation of BRD4 by SETD6 regulates selective transcription to control mRNA translation.

Dzuricky et al. (Nature Chemistry (2020) 12(9) 814-825) disclose systems that use intrinsically disordered proteins to transport dye molecules or alkaline phosphatase into a condensate.

Based on the above-mentioned state of the art, the objective of the present invention is to provide means and methods to study intracellular condensates. This objective is attained by the subject-matter of the independent claims of the present specification, with further advantageous embodiments described in the dependent claims, examples, figures and general description of this specification.

### SUMMARY OF THE INVENTION

One aspect of the invention relates to a system for modifying a target biomolecule that is associated to an endogenous intracellular condensate. The endogenous intracellular condensate may be formed by an intracellular protein comprising an Intrinsically Disordered Region (IDR). The system of the invention comprises as its minimal components an IDR polypeptide sequence tract comprising the Intrinsically Disordered Region of the intracellular protein forming or being associated to the intracellular condensate, and an effector polypeptide domain capable of covalently modifying said target biomolecule.

The invention in one alternative relates to a system for modifying a chemical or physical property of an endogenous intracellular condensate.

Another aspect of the invention relates to a nucleic acid sequence encoding the system according to the previously disclosed aspect of the invention, or encoding a component part of the system.

The invention further relates to a method for modifying a target (particularly a target protein) associated to an endogenous intracellular condensate formed by an intracellular protein comprising an Intrinsically Disordered Region. This method comprises the steps of providing a cell comprising an intracellular protein comprising an Intrinsically Disordered Region, and expressing in said cell a system as specified according to the first aspect of the invention in any of its embodiments.

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising”, “having”, “containing”, and “including”, and other similar forms, and grammatical equivalents thereof, as used herein, are intended to be equivalent in meaning and to be open-ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a”, “or” and “the” include plural referents unless the context clearly dictates otherwise.

“And/or” where used herein is to be taken as specific recitation of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art (e.g., in cell culture, molecular genetics, nucleic acid chemistry, hybridization techniques and biochemistry, organic synthesis). Standard techniques are used for molecular, genetic, and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

The term condensate in the context of the present specification relates to a membrane-less compartment inside of a cell, characterized by different biomolecule concentrations from its cellular surroundings. The term condensate is used synonymously with the term biomolecular condensate, and refers to membrane-less compartments composed of selectively concentrated biomolecules with liquid-like properties. See Hyman et al., Liquid-Liquid Phase Separation in Biology, Annual Review of Cell and Developmental Biology Vol. 30 (2014) pp 39-58; Cho W K, Spille J. et al Science 2018 Jul. 27; 361(6400):412-415; Narayanan et al Elife 2019, https://doi.org/10.7554/eLife.39695, incorporated by reference herein.

Particular embodiments of an endogenous condensate as referenced herein relate to protein condensates. Particular embodiments of an endogenous condensate as referenced herein relate to small sub-diffractive (smaller than 750 nm in diameter) condensates. Particular embodiments of an endogenous condensate as referenced herein relate to transcriptional condensates formed by RNA, transcription factors, RNA polymerase and genomic DNA.

The term endogenous condensate in the context of the present specification relates to a condensate that is present in the cell prior to induction or application of the system according to the invention, in other words, rather than providing the polypeptide components of the condensate through the expression of the fusion polypeptides disclosed herein, the method of the invention targets existing condensates under physiological conditions.

The term effector polypeptide in the context of the present specification relates to a polypeptide inserted into an endogenous intracellular condensate that is capable of chemical or physical property of that endogenous intracellular condensate. In particular embodiments, the effector polypeptide directly or indirectly modifies a target protein by inducing the formation or breakage of a covalent bond.

The term system in the context of the present specification relates to a combination of functional elements that are embodied in polypeptide form and contain one or several IDR tracts and an effector polypeptide at minimum. The system according to the invention can be embodied by a single polypeptide chain, by two or three or even more distinct polypeptide chains that assemble upon expression, or in response to a stimulus.

### Sequences

Sequences similar or homologous (e.g., at least about 70% sequence identity) to the sequences disclosed herein are also part of the invention. In some embodiments, the sequence identity at the amino acid level can be about 80%, 85%, 90%, 91%, 92%, 93%, 94%, 95%, 96%, 97%, 98%, 99% or higher. At the nucleic acid level, the sequence identity can be about 70%, 75%, 80%, 85%, 90%, 91%, 92%, 93%, 94%, 95%, 96%, 97%, 98%, 99% or higher.

In the context of the present specification, the terms sequence identity and percentage of sequence identity refer to a single quantitative parameter representing the result of a sequence comparison determined by comparing two aligned sequences position by position. Methods for alignment of sequences for comparison are well-known in the art. Alignment of sequences for comparison may be conducted by the local homology algorithm of Smith and Waterman, Adv. Appl. Math. 2:482 (1981), by the global alignment algorithm of Needleman and Wunsch, J. Mol. Biol. 48:443 (1970), by the search for similarity method of Pearson and Lipman, Proc. Nat. Acad. Sci. 85:2444 (1988) or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.ncbi.nlm.nih.gov/).

One example for comparison of amino acid sequences is the BLASTP algorithm that uses the default settings: Expect threshold: 10; Word size: 3; Max matches in a query range: 0; Matrix: BLOSUM62; Gap Costs: Existence 11, Extension 1; Compositional adjustments: Conditional compositional score matrix adjustment. One such example for comparison of nucleic acid sequences is the BLASTN algorithm that uses the default settings: Expect threshold: 10; Word size: 28; Max matches in a query range: 0; Match/Mismatch Scores: 1.-2; Gap costs: Linear. Unless stated otherwise, sequence identity values provided herein refer to the value obtained using the BLAST suite of programs (Altschul et al., J. Mol. Biol. 215:403-410 (1990)) using the above identified default parameters for protein and nucleic acid comparison, respectively.

Reference to identical sequences without specification of a percentage value implies 100% identical sequences (i.e. the same sequence).

The term having substantially the same biological activity in the context of the present invention relates to the function described for the specific sequence in the context of the invention. One example is the ability of an IDR tract to facilitate integration of the construct as described in claim 1 of the specification into an endogenous intracellular condensate formed by proteins having the same IDR tract as part of their natural sequence.

### General Biochemistry: Peptides, Amino Acid Sequences

The term polypeptide in the context of the present specification relates to a molecule consisting of 50 or more amino acids that form a linear chain wherein the amino acids are connected by peptide bonds. The amino acid sequence of a polypeptide may represent the amino acid sequence of a whole (as found physiologically) protein or fragments thereof. The term “polypeptides” and “protein” are used interchangeably herein and include proteins and fragments thereof. Polypeptides are disclosed herein as amino acid residue sequences.

The term peptide in the context of the present specification relates to a molecule consisting of up to 50 amino acids, in particular 8 to 30 amino acids, more particularly 8 to 15 amino acids, that form a linear chain wherein the amino acids are connected by peptide bonds.

Amino acid residue sequences are given from amino to carboxyl terminus. Capital letters for sequence positions refer to L-amino acids in the one-letter code (Stryer, Biochemistry, 3^{rd} ed. p. 21). Lower case letters for amino acid sequence positions refer to the corresponding D- or (2R)-amino acids. Sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (lie, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

The term variant refers to a polypeptide that differs from a reference polypeptide, but retains essential properties. A typical variant of a polypeptide differs in its primary amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

In the context of the present specification, the term amino acid linker refers to a polypeptide of variable length that is used to connect two polypeptides in order to generate a single chain polypeptide. Exemplary embodiments of linkers useful for practicing the invention specified herein are oligopeptide chains consisting of 1, 2, 3, 4, 5, 10, 20, 30, 40 or 50 amino acids. A non-limiting example of an amino acid linker is a monomer or di-, tri- or tetramer of a tetraglycine-serine peptide linker.

### General Molecular Biology: Nucleic Acid Sequences, Expression

The term gene refers to a polynucleotide containing at least one open reading frame (ORF) that is capable of encoding a particular polypeptide or protein after being transcribed and translated. A polynucleotide sequence can be used to identify larger fragments or full-length coding sequences of the gene with which they are associated. Methods of isolating larger fragment sequences are known to those of skill in the art.

The term transgene in the context of the present specification relates to a gene or genetic material that has been transferred from one organism to another. In the present context, the term may also refer to transfer of the natural or physiologically intact variant of a genetic sequence into tissue of a patient where it is missing. It may further refer to transfer of a natural encoded sequence the expression of which is driven by a promoter absent or silenced in the targeted tissue.

The term recombinant in the context of the present specification relates to a nucleic acid, which is the product of one or several steps of cloning, restriction and/or ligation and which is different from the naturally occurring nucleic acid. A recombinant virus particle comprises a recombinant nucleic acid.

The terms gene expression or expression, or alternatively the term gene product, may refer to either of, or both of, the processes—and products thereof—of generation of nucleic acids (RNA) or the generation of a peptide or polypeptide, also referred to transcription and translation, respectively, or any of the intermediate processes that regulate the processing of genetic information to yield polypeptide products. The term gene expression may also be applied to the transcription and processing of a RNA gene product, for example a regulatory RNA or a structural (e.g. ribosomal) RNA. If an expressed polynucleotide is derived from genomic DNA, expression may include splicing of the mRNA in a eukaryotic cell. Expression may be assayed both on the level of transcription and translation, in other words mRNA and/or protein product.

The term nucleic acid expression vector in the context of the present specification relates to a plasmid, a viral genome or an RNA, which is used to transfect (in case of a plasmid or an RNA) or transduce (in case of a viral genome) a target cell with a certain gene of interest, or -in the case of an RNA construct being transfected- to translate the corresponding protein of interest from a transfected mRNA. For vectors operating on the level of transcription and subsequent translation, the gene of interest is under control of a promoter sequence and the promoter sequence is operational inside the target cell, thus, the gene of interest is transcribed either constitutively or in response to a stimulus or dependent on the cell's status. In certain embodiments, the viral genome is packaged into a capsid to become a viral vector, which is able to transduce the target cell.

### Binding; Binders, Ligands, Antibodies:

If not specified more narrowly in the Detailed Description of the Invention, reference to binders and ligands encompasses antibodies, antibody-like molecules and aptamers as defined in the following paragraphs.

The term specific binding in the context of the present invention refers to a property of ligands that bind to their target with a certain affinity and target specificity. The affinity of such a ligand is indicated by the dissociation constant of the ligand. A specifically reactive ligand has a dissociation constant of ≤10⁻⁸ mol/L (particularly ≤10⁻⁹ mol/L) when binding to its target, but a dissociation constant at least three orders of magnitude higher in its interaction with a molecule having a globally similar chemical composition as the target, but a different three-dimensional structure.

The term antibody-like molecule in the context of the present specification refers to a molecule capable of specific binding to another molecule or target with high affinity/a Kd≤10⁻⁷ mol/L (particularly ≤10⁻⁹ mol/L). An antibody-like molecule binds to its target similarly to the specific binding of an antibody. The term antibody-like molecule encompasses a repeat protein, such as a designed ankyrin repeat protein (Molecular Partners, Zurich), an engineered antibody mimetic protein exhibiting highly specific and high-affinity target protein binding (see US2012142611, US2016250341, US2016075767 and US2015368302). The term antibody-like molecule further encompasses, but is not limited to, a polypeptide derived from armadillo repeat proteins, a polypeptide derived from leucine-rich repeat proteins and a polypeptide derived from tetratricopeptide repeat proteins. The term antibody-like molecule further encompasses a specifically binding polypeptide derived from a protein A domain, a fibronectin domain FN3, a consensus fibronectin domain, a lipocalin (see Skerra, Biochim. Biophys. Acta 2000, 1482(1-2):337-50), a polypeptide derived from a Zinc finger protein (see Kwan et al. Structure 2003, 11(7):803-813), a Src homology domain 2 (SH2) or Src homology domain 3 (SH3), a PDZ domain, a gamma-crystallin, ubiquitin, a cysteine knot polypeptide or a knottin, cystatin, Sac7d, a triple helix coiled coil (also known as alphabodies), a Kunitz domain or a Kunitz-type protease inhibitor and a carbohydrate binding module 32-2. The term antibody-like molecule further encompasses a humanized camelid antibody. The term antibody-like molecule similarly encompasses an scFv fragment.

The term protein A domains derived polypeptide refers to a molecule that is a derivative of protein A and is capable of specifically binding the Fc region and the Fab region of immunoglobulins.

The term armadillo repeat protein refers to a polypeptide comprising at least one armadillo repeat, wherein an armadillo repeat is characterized by a pair of alpha helices that form a hairpin structure.

In the context of the present specification, the term fragment crystallizable (Fc) region is used in its meaning known in the art of cell biology and immunology; it refers to a fraction of an antibody comprising, if applied to IgG, two identical heavy chain fragments consisting of a C_{H}2 and a C_{H}3 domain, covalently linked by disulfide bonds.

In the context of the present specification, the term single-chain variable fragment (scFv) relates to a fusion protein of the variable regions of the heavy (VH) and light chains (VL) of an immunoglobulin, resulting in an antibody-like, high affinity to the target from a single polypeptide chain. The VH and VL chains of the scFv are connected through a short linker peptide of ten to about 25 amino acids [Huston et al. (1988). PNAS 85 (16): 5879-5883] The linker can either connect the N-terminus of the VH with the C-terminus of the VL (VL-VH), or adopt the reverse configuration (VH-VL).

### DETAILED DESCRIPTION OF THE INVENTION

### The System

A first aspect of the invention relates to a system for modifying a chemical or physical property of an endogenous intracellular condensate.

As a specific example of such system is a system for modifying a target biomolecule, wherein the target biomolecule is associated to an endogenous intracellular condensate formed by an intracellular protein comprising an Intrinsically Disordered Region (IDR).

The system comprises at least two components, and IDR tract and an effector, that are associated on a single polypeptide chain. Alternatively, the at least to components are each located on distinct polypeptide chains that associate to one molecular complex either spontaneously or in response to an external stimulus, such as the irradiation with light, or the cessation of irradiation by light.

The components of the system can also be present in a system comprised of at least three parts wherein an adaptor polypeptide facilitates binding of the IDR part to the effector part in different ratios.

The system may be comprised of more than one IDR and/or more than one effector, facilitating different ratios of IDR to effector, either more IDR per effector, or more effector per IDR.

The two minimal components of the system are:
a. an IDR polypeptide sequence tract comprising said Intrinsically Disordered Region and
b. an effector polypeptide domain capable of covalently modifying said target.

In order for the IDR tract to fit into the condensate, not the entire IDR region of the condensate-associated protein might need to be comprised in the IDR tract.

In certain particular embodiments, the components of the system are expressed as transgenes in a cell, for example from an artificial expression plasmid encoding the components.

In specific embodiments, the target is a target biomolecule.

In certain embodiments, the system comprises one effector domain and more than one IDR sequence tracts. More than one IDR may be required to allow the entry of bulky or complex protein “payloads” into a condensate.

In certain particular embodiments, the system comprises one effector domain and 2, 3, 4 or ≥5 IDR sequence tracts.

In certain embodiments, the system comprises one effector domain and one IDR sequence tract.

In certain embodiments, the system comprises one IDR sequence tract and more than one effector domains.

### Intrinsically Disordered Regions

According to a definition cited by Madan Babu (Biochem Soc Trans. 2016 Oct. 15; 44(5): 1185-1200),
Intrinsically disordered regions (IDRs) are polypeptide segments that do not contain sufficient hydrophobic amino acids to mediate co-operative folding. Instead, they typically contain a higher proportion of polar or charged amino acids [Uversky et al. Proteins: Struct., Funct., Bioinf. 41, 415-427]. Thus, IDRs lack a unique three-dimensional structure either entirely or in parts in their native state. They generally sample a variety of conformations that are in dynamic equilibrium under physiological conditions [Forman-Kay and Mittag (2013), Structure 21, 1492-1499, 32-34].

In certain embodiments the protein comprising an IDR is a protein located in the nucleus of a eukaryote cell.

The embodiments shown in the Examples show experiments performed in eukaryote cells, in the nucleus, on transcription-associated condensates. The invention however is not limited to application of the system in nuclear condensates, or even in eukaryotes.

A number of physiologically relevant proteins comprising IDR have been identified. For the purposes of defining IDR in the context of the present invention, the IDRs assigned for the sequences of proteins comprised in the following list are considered embodiments of the IDRs useful in practicing the invention. The invention is not, however, to be considered limited to the IDRs of proteins contained in this list.

In certain embodiments, the protein comprising an IDR is selected from the group comprised of the proteins: BRD4, NELFA, NELFB, CDK9, P-TEFb, Mediator complex; RNA Polymerase (RPB1; Uniprot ID P24928)

In certain particular embodiments, the protein comprising an IDR is selected from the group comprised of BRD4, NELFA, NELFB, CDK9, P-TEFb, RPB1

In certain more particular embodiments, the protein comprising an IDR is selected from more BRD4 and NELFA;

In certain embodiments, the IDR tract is derived of the IDR of BRD4 (Uniprot ID 060885).

In certain particular embodiments, the IDR sequence tract is selected from SEQ ID NO 001 and SEQ ID NO 002 (ΔN-BDR4 IDR; NELFA IDR), or a sequence variant thereof characterized by at least 85% sequence identity to any of (sequences previously recited), said variant having the biological function of facilitating association of the system according to the invention with the endogenous condensate.

In certain embodiments, the IDR tract is derived of the IDR of NELFA (Negative Elongation Factor Complex Member A; Uniprot ID Q9H3P2).

In certain embodiments, the IDR tract is derived of the IDR of NELFB (Negative Elongation Factor Complex Member B; Uniprot ID Q8WX92).

In certain embodiments, the IDR tract is derived of the IDR of CDK9 (Cyclin-dependent kinase 9; Uniprot ID P50750).

In certain embodiments, the IDR tract is derived of the IDR of Cyclin T1 (CCNT1, Uniprot 060563).

In certain embodiments, the IDR tract is derived of the IDR of Human MED1 CTD (Uniprot ID Q15648).

In certain embodiments, the IDR tract is derived of the IDR of Pol II catalytic subunit RPB1 C-terminal domain (CTD) (RPB1 IDR; Uniprot ID P24928)

In certain embodiments, the protein comprising an IDR is selected from the following list:

| Protein | Biological role | Reference |
|---|---|---|
| MED1 | coactivator overexpressed | (Cho et al., (2018) Science 361(6400), |
| | and modified in cancer | 412-415; Nagalingam et al., (2012) |
| | | Carcinogenesis 33(4), 918-930; Russo et |
| | | al., (2019) Cancer Discov. 9(11), 1490- |
| | | 1492; Sabari et al., (2018) Science |
| | | 361(6400), eaar3958;) |
| RPB1 | Subunit of RNA polymerase II | (Cho et al., (2018) Science 361(6400), |
| | | 412-415;) |
| CDK7 | kinase overexpressed and | (Klein et al., (2020) Science 368(6497), |
| | targeted in cancer | 1386-1392; Kwiatkowski et al., (2014) |
| | | Nature 511, 616-620;) |
| P-TEFb | kinase implicated in ovarian | (Guo et al., (2019) Nature 572, 543-548; |
| | cancer | Kohoutek, (2009) Cell Div. 4, 19;) |
| YAP/TAZ | coregulator with increased | (Cai et al., (2019) Nat. Cell Biol. 21, |
| | activity in various cancers | 1578-1589; Zanconato et al., (2018) |
| | | Nat. Med. 24(10), 1599-1610;) |
| EWS | fused to FLI in Ewing's | (Boulay et al., (2017) Cell 171(1), |
| | sarcoma | 163-178; Chong et al., (2018) Science |
| | | 361(6400), eaar2555) |
| OCT4 | master TF regulator of cell | (Boija et al., (2018) Cell 175(7), |
| | identity | 1842-1855;) |
| HSF1 | TF overexpressed in cancer | (Carpenter and Go′kmen-Polar, (2018) |
| | | Curr. Cancer Drug Targets 19, 515-524; |
| | | Gaglia et al., (2020) Nat. Cell Biol. 22, |
| | | 151-158;) |
| FUS | translocated in sarcoma | (Crozat et al., (1993) Nature 363, 640-644; |
| | | Kato et al., (2012) Cell 149(4), 753-767;) |
| MYC | overexpressed in cancer | (Boija et al., (2018) Cell 175(7), 1842-1855; |
| | | Dang, (2012) Cell 149(1) 22-35;) |
| TAF15 | cofactor implicated in cancer | (Altmeyer et al., 2015; Kwon et al., 2013 |
| | | Shin et al., (2018) Cell 175(6), 1481-1491; |
| | | Wei et al., 2020;) |
| ENL | cofactor translocated in | (Wan et al., (2017) Nature 543, 265-269; |
| | leukemia | Wan et al., (2020) Nature 577(7788), |
| | | 121-126;) |
| BRD4 | chromatin factor upregulated | (Filippakopoulos et al., (2010) Nature |
| | and fused in cancer | 468(7327), 1067-1073; Sabari et al., (2018) |
| | | Science 361(6400), eaar3958; Shin et al., |
| | | (2018) Cell 175(6), 1481-1491;) |
| HP1a | chromatin factor downregulated | (Larson et al., (2017) Nature 547, 236-240; |
| | in cancer | Strom et al., (2017) Nature 547, 241-245; |
| | | Vad-Nielsen and Nielsen, (2015) Cancer |
| | | Biol. Ther. 16, 189-200;) |
| DAXX | 1. Transcriptional regulation. | (Mahmud and Liao, (2019), Nucleic Acids |
| | 2. DNA repair, and respond in | Res. 47(15), 7734-7752; Tong et al., (2022) |
| | viral infection. | Signal Transduct. Target Ther. 7, 221;) |
| | 3. Impact apoptosis and cell | |
| | signaling. | |
| 53BP1 | DNA repair factor and tumor | (Kilic et al., (2019) EMBO J. 38, e101379; |
| | suppressor | Mirza-Aghazadeh-Attari et al., (2019) DNA |
| | | Repair (Amst) 73, 110-119; Pryde et al., |
| | | (2005) J. Cell Sci. 118(9), 2043-2055;) |
| FUS, | tress granules are antiapoptotic | (Arimoto et al., (2008) Nat. Cell Biol. |
| hnRPNA1, | | 10(11), 1324-1332; Molliex et al., (2015) |
| G3NP1/2 | | Cell, 163(1), 123-124; Protter and Parker, |
| | | (2016) Trends Cell Biol. 26(9), 668-679;) |
| TAU | Neurodegenerative diseases | (Wegmann et al., (2018) EMBO J. 37(7), |
| | (Alzheimer's diseases) | e98049;) |
| a-synuclein | Neurodegenerative diseases | (Ray et al., (2020) Nat. Chem. 12(8), |
| | (Parkinson diseases) | 705-716;) |
| MED1 | coactivator overexpressed and | (Cho et al., 2018; Nagalingam et al., 2012; |
| | modified in cancer | Russo et al., 2019; Sabari et al., 2018) |
| CDK7 | kinase overexpressed and | (Klein et al., 2020; Kwiatkowski et al., |
| | targeted in cancer | 2014) |
| P-TEFb | kinase implicated in ovarian | (Guo et al., 2019; Kohoutek, 2009) |
| | cancer | |
| YAP/TAZ | coregulator with increased | (Cai et al., 2019a; Zanconato et al., 2018) |
| | activity in various cancers | |
| EWS | fused to FLI in Ewing's | (Boulay et al., 2017; Chong et al., 2018) |
| | sarcoma | |
| OCT4 | master TF regulator of cell | (Boija et al., 2018) |
| | identity | |
| HSF1 | TF overexpressed in cancer | (Carpenter and Go′kmen-Polar, 2018; |
| | | Gaglia et al., 2020) |
| FUS | translocated in sarcoma | (Crozat et al., 1993; Kato et al., 2012) |
| MYC | overexpressed in cancer | (Boija et al., 2018; Dang, 2012) |
| TAF15 | cofactor implicated in cancer | (Altmeyer et al., 2015; Kwon et al., 2013; |
| | | Shin et al., 2018; Wei et al., 2020) |
| ENL | cofactor translocated in | (Wan et al., 2017, 2020) |
| | leukemia | |
| BRD4 | chromatin factor upregulated | (Filippakopoulos et al., 2010; Sabari et |
| | and fused in cancer | al., 2020; Shin et al., 2018) |
| HP1a | chromatin factor downregulated | (Larson et al., 2017; Strom et al., 2017; |
| | in cancer | Vad- Nielsen and Nielsen, 2015) |
| DAXX | 1. Transcriptional regulation. | (Cai et al., 2021;) |
| | 2. DNA repair, and respond in | |
| | viral infection. | |
| | 3. Impact apoptosis and cell | |
| | signaling. | |
| 53BP1 | DNA repair factor and tumor | (Kilic et al., 2019; Mirza-Aghazadeh-Attari |
| | suppressor | et al., 2019; Pryde et al., 2005) |
| FUS, | tress granules are antiapoptotic | (Arimoto et al., 2008; Molliex et al., |
| hnRPNA1, | | 2015; Protter and Parker, 2016) |
| G3NP1/2 | | |
| TAU | Neurodegenerative diseases | (Wegmann et al., 2018) |
| | (Alzheimer's diseases) | |
| a-synuclein | Neurodegenerative diseases | (Ray et al., 2020) |
| | (Parkinson diseases) | |

The person skilled in the art will be able to identify further proteins of interest to practice the invention from the following review articles, incorporated herein by reference:
Tong et al., Liquid-liquid phase separation in tumor biology; Nature Signal Transduction and Targeted Therapy 7, Art. No 221 (2022);
Boja et al. 2021; Biomolecular Condensates and Cancer, Cancer Cell 39, 174-192;
Cai et al. 2021; Biomolecular Condensates and Their Links to Cancer Progression; Trends in Biochemical Sciences 46, 535-549;
Zbinden et al. 2020; Phase Separation and Neurodegenerative Diseases: A Disturbance in the Force, Developmental Cell 55 45-68.

Prediction of IDR: Disorder can be predicted on the basis of protein sequence information. The inventors used the PONDR software available at pondr.com (Predictor of Natural Disordered Regions) and the derived VSL2 score as an output determinator. Peng et al., (2006) BMC Bioinformatics 7:208 incorporated herein by reference). A sequence tract of 50 or more consecutive amino acid (AA) positions above a VSL2 score equal to or above (≥) 0.5 is considered to be an IDR tract suitable for practicing the invention.

In certain embodiments, the IDR tract is a stretch of 50 AA characterized by VSL2 score≥0.7.

In certain more particular embodiments, IDR tract is a stretch of 50 AA characterized by VSL2 score≥0.8.

In certain embodiments, the IDR tract is a stretch of 100 AA characterized by VSL2 score≥0.5.

In certain particular embodiments, IDR tract is a stretch of 100 AA characterized by VSL2 score≥0.7.

In certain more particular embodiments, the IDR tract is a stretch of 100 AA characterized by VSL2 score≥0.8.

In certain even more particular embodiments, IDR tract is a stretch of 50 AA characterized by VSL2 score≥0.9.

In the embodiments shown in the examples, the “natural” IDR and the probe (FP1) IDR overlap 100%. Without wanting to be bound by theory, the inventors assume that IDR tracts having a similar “chemical grammar” will be assembled into the same condensates. The inventors have not determined in detail what percentage of overlap has to exist between a “natural” IDR and a shortened IDR sequence tract for the latter to be permitted into the condensates.

In certain embodiments, the IDR sequence tract comprises at least (≥) 80% of the IDR of a natural condensate forming (Intrinsically Disordered) protein. In certain particular embodiments, the IDR sequence tract comprises ≥85% of the IDR of a natural condensate forming protein. In certain more particular embodiments, this percentage is ≥90%. In certain even more particular embodiments, this percentage is ≥95% or even ≥98%.

### The Target

In general, the system of the invention targets condensates, in the sense that it facilitates the manipulation of condensates by allowing condensate-specific addition of polypeptides, either by controlled expression of the system of the invention, or by stimulus-dependent association of the IDR and effector part of the system. The effect may consist in simply disturbing a chemical or physical balance within the condensate, leading to a change in its physiological behaviour.

In a practical sense, an immediate application illustrated by the examples contained herein is the introduction of an enzymatic effector that allows covalent modification of proteins associated with the condensate (either actively forming the condensate or being contained or functionally associated thereto).

In certain embodiments, a target biomolecule is a protein. In a particular embodiment, the target biomolecule is an intracellular protein. In a more particular embodiment, the target biomolecule is an intracellular nuclear protein. In an even more particular embodiment, the target biomolecule is a nuclear protein associated to RNA polymerase II activity.

The target type addressed in the Examples is protein; however, targets for manipulation or modification are not limited to proteins but also include DNA and RNA, which are found inside of biomolecular condensates. As one non-limiting example, if DamID is used as the effector modality, it will modify (methylate) DNA.

### The Effector

In parallel to what is said about the target in its most general abstraction in the preceding paragraphs, the effector polypeptide can be regarded as a functionality that facilitates disturbing a chemical or physical balance within the condensate, leading to a change in its physiological behaviour.

In particular embodiments, the effector is a functionality that facilitates covalent modification of condensate-associated biomolecules by inducing the formation or breakage of a covalent bond.

In certain particular embodiments, the effector polypeptide is a polypeptide capable of biotinylating a target, particularly a target protein.

In certain particular embodiments, the effector polypeptide is a polypeptide capable of ubiquitinylating a target, particularly a target protein.

In certain particular embodiments, the effector polypeptide is a polypeptide capable of methylating a target, particularly a target protein.

In certain particular embodiments, the effector polypeptide is a polypeptide capable of demethylating a target, particularly a target protein.

In certain particular embodiments, the effector polypeptide is a polypeptide capable of acetylating a target, particularly a target protein.

In certain particular embodiments, the effector polypeptide is a polypeptide capable of deacetylating a target, particularly a target protein.

In certain particular embodiments, the effector polypeptide is a polypeptide capable of phosphorylating a target, particularly a target protein.

In certain particular embodiments, the effector polypeptide is a polypeptide capable of dephosphorylating a target, particularly a target protein.

In particular embodiments, the effector polypeptide is a biotin activating agent characterized by SEQ ID NO 007 or a variant thereof having at least 50% of its activity. The activity of this construct is disclosed in Branon et al., (2018) Nat. Biotechnol., 36(9):880-887, which is incorporated herein by reference.

Various enzymatic activities may be employed to modify target moieties in the endogenous condensate. The inventors employed the so-called BioID proximity-based labelling system, an E. coli-derived biotin ligase (BirA*) characterized by a catalytic site mutation (R118G) that destabilizes retention of an activated biotin molecule (biotinoyl-5′-AMP), which dissociates from the ligase and reacts with primary amines of exposed lysine residues in neighbouring proteins, resulting in covalent attachment of biotin to the target (see Trinkle-Mulcahy, F1000Research 2019, 8(F1000 Faculty Rev):135 and references cited therein). Variants of this system are marketed under the designations TurboID, split TurboID, BioID2, BASU and APEX. Another activity for modifying/tagging proteins is ubiquitin ligase.

An alternative activity for modifying DNA is DNA adenine methyltransferase, marketed as DamID.

One advantage of the method and components for using the method of the invention is its versatility. Biotinylation of a target structure is just one possible application. Different IDR can target different types of endogenous condensates, and different “cargo”, i.e. modifying effectors, can modify the condensates differently.

### Separation of IDR and Effector to Different Polypeptide Constructs

In certain embodiments, the DR sequence tract and the effector domain are located on distinct polypeptide molecules that can be induced to associate in response to a stimulus. In certain embodiments, the stimulus causes the association of the distinct polypeptide molecules that contain the DR sequence tract and the effector domain, respectively. These distinct polypeptide molecules are present prior to the stimulus causing their association.

In certain embodiments, this stimulus is light.

In certain embodiments, association of a first fusion polypeptide comprising the DR tract and another fusion peptide comprising the effector domain is facilitated by a light-induced binding partner pair. The light-induced binding partner pair may consist of a first binding partner and a second binding partner, and the first and second binding partners associate (or dissociate) in presence of light. To effectively allow control over the generation of the system of the invention, one of the binding partners is part of a fusion polypeptide comprising an IDR, and the other of the binding partners is directly or indirectly associated with the effector domain.

In certain embodiments, the light-induced binding partner pair is selected from the group comprised of:
SspB-iLID, ΔPhyA-FHY-1 and ΔPhyA-FHL (association: 660 nm/dissociation: dark or 740 nm; Zhou et al., Nature Biotechnology 40, 262-272 (2022);
PhyB/PIF3 and PhyB/PIF6 (association: 660 nm/dissociation: dark or 740 nm; Toettcher et al., Nature Methods 8, 837-839 (2011));
UVR8/COP1 (300 nm; Crefcoeur et al., Nature Communications 4, Article: 1779 (2013));
CRY2/CIB1 (450 nm; Konermann et al. Nature 500, 472-476 (2013));
FKF1/GI (450 nm; Yazawa et al., Nature Biotechnology 27, 941-945 (2009));
VVD variants of Neurospora crassa termed Magnets (450 nm; Kawano et al. Nature Communications 6, Article: 6256 (2015));
AsLOV2—ePDZ (“Tulip”; Strickland et al., Nature Methods 9, 379-384 (2012));
cpLOV2-SspB (He et al., Nature Chemical Biology 17, 915-923 (2021));
BphP1/PpsR2 (association: 760 nm/dissociation: dark or 640 nm; Kaberniuk et al., Nature Methods 13, 591-597 (2016));
BphP1/Q-PAS1 (association: 760 nm/dissociation: dark or 640 nm, Redchuk et al., Nature Chemical Biology 13, 633-639 (2017));
MagRed (DrBphP/Aff6_V17FΔN, association: 660 nm/dissociation: dark or 780 nm, Kuwasaki et al., Nature Biotechnology 40, 1672-1679 (2022)).

The use of “optogenetic photoswitches” which enable association of protein units as a consequence of irradiation with light of a specific wavelength is well established in the art, as evidenced by the many examples for such light-induced binding partner pairs above. Furthermore, binding pairs are known where dissociation is caused by light, for example as reported by Karapinar et al., Nature Communications 12, Article: 4488 (2021). Thus, while the examples contained herein focus on the establishment of the association as a consequence of light, the reverse mechanism may also be used.

In certain particular embodiments, the light induced binding partner pair is SspB and LID (SEQ ID NO 003 and 004) derived from the light-oxygen-voltage 2 (LOV2) domain from Avena sativa (Guntas et al. (2015) PNAS 112, 112-117). This is the system used in the examples.

### a System Comprised of Three Components, Referred to as LITEC Herein

In certain particular embodiments, the system according to the invention consists of:
a. a plurality of a first fusion polypeptide comprising an IDR sequence tract comprising [at least 80% of] the Intrinsically Disordered Region, an optional first fluorescence marker polypeptide, and a first component of a light-induced binding partner pair;
b. a plurality of a second fusion polypeptide comprising a second component of the light-induced binding partner pair, an optional second fluorescence marker polypeptide and a binding domain capable of specifically binding to a non-endogenous peptide epitope;
c. a third fusion polypeptide comprising a plurality of said non-endogenous peptide epitopes and an effector polypeptide domain capable of covalently modifying said target.

### The First Fusion Polypeptide

The first fusion polypeptide (FP1) is a sequence distinct from the second and third fusion polypeptide that interacts with the second fusion polypeptide. Association to and uptake into an endogenous condensate is mediated by the IDR sequence tract, which is also a “natural” part, being comprised in its constituent proteins, of the condensate.

FP1 contains the IDR tract and a partner of a light-induced binding pair. In the examples, the IDR is on the N terminal section of the FP1 polypeptide, whereas the binding partner is on the C terminal section. In certain embodiments, this polarity can be reversed, in other words the IDR can be on the C terminal end and the binding partner on the N terminal end.

The examples show FP1 constructs comprising a first fluorescence marker polypeptide, specifically the “mCherry” red fluorescent protein derived from the DsRed protein of Discosoma. Other fluorescent proteins may be used. The fluorescence or fluorescent marker polypeptide is an optional component that facilitates visualization of the process of assembly and condensate uptake, but in the opinion of the inventors, is not essential for practicing the invention.

In certain particular embodiments, the first fusion polypeptide does not comprise a fluorescence marker polypeptide.

In certain other particular embodiments, the first and/or second polypeptide comprises a fluorescence marker polypeptide selected from mCherry and eGFP.

In certain particular embodiments, the first fusion polypeptide is characterized by SEQ ID NO 008.

In certain particular embodiments, the first fusion polypeptide is characterized by SEQ ID NO 009.

In certain particular embodiments, the first fusion polypeptide is characterized by SEQ ID NO 010.

In certain particular embodiments, the first fusion polypeptide is characterized by SEQ ID NO 011.

### The Second Fusion Polypeptide

The second fusion polypeptide contains a second binding partner of the light-induced binding pair, the first partner of which is part of the first fusion polypeptide.

The second fusion polypeptide (FP2) also contains a binding domain capable of specifically binding to a non-endogenous peptide epitope. This quality of binding to a non-physiological epitope enables the specific assembly of the FP2 on a string of epitopes introduced into a cell, without distraction by endogenous binding partners and interference with whatever physiological function these binding partners might have.

In certain particular embodiments, the binding domain capable of specifically binding to a non-endogenous peptide epitope is an scFv (single chain variable) antibody fragment.

A particular example of such binding domain is a scFv fragment as used in the Examples. However, any other antibody-like construct as known in the art can be used.

The second binding partner of the light-induced binding partner pair and the binding domain capable of specifically binding to a non-endogenous peptide epitope are advantageously located on either end of the FP2 construct. In one embodiment, the second binding partner of the light-induced binding partner pair is on the N-terminus, and the binding domain capable of specifically binding to a non-endogenous peptide epitope is on the C-terminus. This order could be reversed.

In certain particular embodiments, the non-endogenous peptide epitope is SEQ ID NO 005.

In certain particular embodiments, the non-endogenous peptide epitope is comprised as multiple copies in a construct exemplified by SEQ ID NO 006.

The examples show FP2 constructs comprising a second fluorescence marker polypeptide, specifically a green fluorescent protein derived from the Aequorea victoria. Other fluorescent proteins may be used. As stated above, the fluorescence or fluorescent marker polypeptides comprised on the first and/or second constructs are optional components that facilitate visualization of the process of assembly and condensate uptake, but in the opinion of the inventors, are not essential for practicing the invention.

In certain particular embodiments, the second fusion polypeptide does not comprise a fluorescence marker polypeptide.

In certain particular embodiments, the second fusion polypeptide is characterized by SEQ ID NO 012 (sequence of scFv-GFP-iLID and, if feasible, scFv-iLID).

### The Third Fusion Polypeptide

The third fusion polypeptide FP3 comprises the enzymatic function for modifying target moieties in the condensate, and a number of attachment/binding sites (“epitopes”) for the binding domain comprised in the second fusion polypeptide.

The attachment sites for the binding domains of the FP2 construct serve as multipliers of the signal conveyed by the IDR regions, facilitating the integration of large entities into the condensate.

In addition to scFv/SunTag system employed in the examples, a great number of tagging system exist, many of them developed to facilitate affinity purification or fluorescent protein signal amplification. These systems include, but are not limited to, the MoonTag, FLAG Tag, TAP tag and many more.

In certain embodiments, the FP3 construct comprises 3 to 40 copies of the epitope, particularly 4 to 30, more particularly 10 to 20 multiple copies of the same peptide epitope of 6 to 15 amino acids in length. In particular embodiments, the epitope sequence is selected to not be part of the expressed genome of the cell in which the condensate is present (i.e., in which the system is to be used), in order to avoid cross-reaction of the epitope's binder with cellular structures.

In certain particular embodiments, the third fusion polypeptide is characterized by SEQ ID NO 13 (sequence of bioID-SunTag).

### Nucleic Acids and Vectors

Another aspect of the invention relates to a nucleic acid sequence, or a plurality of nucleic acid sequences, encoding the system as specified in any of the above discussed aspects of the invention. The embodiments characterizing distinct features of the system may be encoded in the nucleic acid sequence.

One particular embodiment of this aspect relates to a nucleic acid sequence encoding the third fusion polypeptide as specified above.

The nucleic acid according to this aspect maybe comprised in an expression vector, for example a DNA plasmid, or a viral vector. Alternatively, the nucleic acid may be one or several mRNA molecules encoding the system. Parts of the system may be encoded on a plasmid vector for easy insertion of, for example, different IDR or effector components that facilitate fast and easy insertion of protein components to be employed as part of the system.

### Methods

Another aspect of the invention relates to a method for modifying or manipulating a chemical or physical property of an endogenous intracellular condensate.

In specific embodiments of this aspect, the method is employed to modifying a target, particularly a target protein, associated to an endogenous intracellular condensate formed by an intracellular protein comprising an Intrinsically Disordered Region. This method comprises the steps:
a) providing a cell comprising an intracellular protein comprising an Intrinsically Disordered Region;
b) expressing in said cell a system as specified in any of the above discussed aspects of the invention.

In certain particular embodiments, the method additionally comprises the steps of
c) collecting or isolating a preparation comprising target biomolecules from said cell,
d) isolating target biomolecules modified by said effector polypeptide capable of covalently modifying the target biomolecules.

In certain particular embodiments, the effector polypeptide is capable of biotinylating target biomolecules, and isolating biotinylated target biomolecules is attained by binding biotinylated proteins from the preparation comprising biomolecules to a matrix.

Isolated target biomolecules may subsequently be identified. One particular method for identification proven as useful for this objective is mass spectrometry.

In certain more particular embodiments, the target biomolecules are proteins.

Wherever alternatives for single separable features such as, for example, an IDR tract, light-induced binding partner pair or effector polypeptide, are laid out herein as “embodiments”, it is to be understood that such alternatives may be combined freely to form discrete embodiments of the invention disclosed herein. Thus, any of the alternative embodiments for an IDR tract may be combined with any of the alternative embodiments of effector polypeptide and these combinations may be combined with any fluorescence marker mentioned herein.

The invention further encompasses the following items:
1. A system for modifying a target,
   the target being associated to an endogenous intracellular condensate formed by an intracellular protein comprising an Intrinsically Disordered Region (IDR),
   or a system for modifying a chemical or physical property of an endogenous intracellular condensate,
   said system comprising:
   a. an IDR sequence tract comprising said Intrinsically Disordered Region and
   b. an effector domain capable of modifying said target.
2. The system according to item 1, wherein the target is modified by covalent modification.
3. The system according to item 1 or 2, wherein the protein comprising an IDR is a protein located in the nucleus of a eukaryote cell.
4. The system according to any one of the preceding items, wherein the protein comprising an IDR is selected from the group comprised of the proteins: BRD4, NELFA, NELFB, CDK9, P-TEFb, Mediator complex; RNA Polymerase (RPB1);
   particularly wherein the protein comprising an IDR is selected from the group comprised of BRD4, NELFA, NELFB, CDK9, P-TEFb, RPB1;
      more particularly wherein the protein comprising an IDR is BRD4 or NELFA;
         even more particularly wherein the protein comprising an IDR is BRD4.
5. The system according to any one of the preceding items, wherein the IDR sequence tract is selected from SEQ ID NO 001 and SEQ ID NO 002 (ΔN-BDR4 IDR; NELFA IDR), or a sequence variant thereof characterized by at least 85% sequence identity to any of SEQ ID NO 001 and SEQ ID NO 002.
6. The system according to any one of the preceding items, wherein the system comprises one effector domain and more than one IDR sequence tracts,
   particularly wherein the system comprises 2, 3, 4 or ≥5 IDR tracts.
7. The system according to any one of the preceding items 1 to 5, wherein the system comprises one effector domain and one IDR sequence tract.
8. The system according to any one of the preceding items 1-5, wherein the system comprises one IDR sequence tract and more than one effector domains.
9. The system according to any one of the preceding items, wherein the target is a protein, particularly an intracellular protein, more particularly an intracellular nuclear protein, even more particularly a nuclear protein associated to RNA polymerase II activity.
10. The system according to any one of the preceding items, wherein the effector polypeptide capable of covalently modifying said target is selected from a polypeptide capable of
   a. biotinylating,
   b. ubiquitinylating,
   c. methylating,
   d. demethylating,
   e. acetylating,
   f. deacetylating,
   g. phosphorylating,
   h. dephosphorylating
   a target.
11. The system according to any one of the preceding items, wherein the IDR sequence tract and the effector domain are located on distinct polypeptide molecules that can be induced to associate in response to a stimulus.
12. The system according to item 11, wherein the stimulus is light.
13. The system according to item 12, wherein association of a first fusion polypeptide comprising the IDR tract and another fusion peptide comprising the effector domain is facilitated by a light-induced binding partner pair,
   the light-induced binding partner pair consisting of a first binding partner and a second binding partner, wherein the first and second binding partners associate in presence of light,
   and wherein one of the binding partners is part of the first fusion polypeptide and the other of the binding partners is associated with the effector domain.
14. The system according to item 13, wherein said light-induced binding partner pair is selected from the group comprised of:
   SspB-iLID, ΔPhyA-FHY-1 and ΔPhyA-FHL;
   PhyB/PIF3 and PhyB/PIF6;
   UVR8/COP1; CRY2/CIB1;
   FKF1/GI;
   VVD variants of Neurospora crassa;
   AsLOV2—ePDZ;
   cpLOV2-SspB;
   BphP1/PpsR2;
   BphP1/Q-PAS1;
   MagRed.
15. The system according to item 13 or 14, wherein the light induced binding partner pair is SspB and LID (SEQ ID NO 003 and 004).
16. The system according to any one of the preceding items, wherein
   the system consists of:
      a. a first fusion polypeptide comprising an IDR sequence tract comprising said Intrinsically Disordered Region, [an optional first fluorescence marker polypeptide] and a first component of a light-induced binding partner pair;
      b. a second fusion polypeptide comprising a second component of said light-induced binding partner pair, [an optional second fluorescence marker polypeptide] and a binding domain capable of specifically binding to a non-endogenous peptide epitope;
      c. a third fusion polypeptide comprising a plurality of said non-endogenous peptide epitopes and an effector domain capable of covalently modifying said target.
17. The system according to item 14, wherein said binding domain capable of specifically binding to a non-endogenous peptide epitope is an scFv (single chain variable) antibody fragment.
18. The system according to any one of the preceding items 16 or 17, wherein the non-endogenous peptide epitope is SEQ ID NO 005.
19. The system according to any one of the preceding items 16 to 18, wherein the first fusion polypeptide does not comprise a fluorescence marker polypeptide.
20. The system according to any one of the preceding items 16 to 19, wherein the second fusion polypeptide does not comprise a fluorescence marker polypeptide.
21. The system according to any one of the preceding items 16 to 18, wherein said first fusion polypeptide is characterized by a sequence selected from SEQ ID NO 008, 009, 010, 011.
22. The system according to any one of the preceding items 16 to 18, wherein said second fusion polypeptide is characterized by SEQ ID NO 012 (sequence of scFv-GFP-iLID and, if feasible, scFv-iLID).
23. The system according to any one of the preceding items 16 to 22, wherein said third fusion polypeptide is characterized by SEQ ID NO 13 (sequence of bioID-SunTag).
24. A nucleic acid sequence, or a plurality of nucleic acid sequences, encoding the system as specified in any one of items 1 to 22.
25. A nucleic acid sequence encoding the third fusion polypeptide as specified in item 23.
26. An expression vector encoding a system or a polypeptide component of the system according to any one of items 1 to 23.
   particularly wherein the expression vector is selected from the group of a plasmid, an RNA expression vector, and a virus.
27. A method for modifying a target (particularly a target protein) associated to an endogenous intracellular condensate formed by an intracellular protein comprising an Intrinsically Disordered Region, said method comprising the steps:
   a) providing a cell comprising an intracellular protein comprising an Intrinsically Disordered Region;
   b) expressing in said cell a system as specified in any one of items 1 to 23.
28. The method according to item 27, wherein the method further comprises the steps of
   c) collecting a preparation comprising target biomolecules (particularly proteins) from said cell,
   d) isolating target biomolecules (particularly proteins) modified by said effector polypeptide capable of modifying the target biomolecules.
29. The method according to item 27 or 28, wherein the effector polypeptide is capable of biotinylating target biomolecules, and isolating biotinylated target biomolecules is attained by binding biotinylated proteins from the preparation comprising biomolecules to a matrix.
30. The method according to item 28 or 29, wherein isolated target biomolecules are identified, particularly by mass spectrometry.

The invention is further illustrated by the following examples and figures, from which further embodiments and advantages can be drawn. These examples are meant to illustrate the invention but not to limit its scope.

### DESCRIPTION OF THE FIGURES

FIG. 1 Intrinsically disordered regions (IDRs) target the endogenous transcriptional condensates. (A and B) Intrinsically disordered regions of BRD4 and NELFA are marked as bold black bars using a PONDR VSL2 algorithm, respectively. Y-axis is VSL2 scores and X-axis is amino acid (aa) positions. For BRD4, IDR part (462nd aa to 1362nd aa) of BRD4 is used. For NEFLA, the whole amino acid of NELFA is used. (C-F) IDR of BRD4 colocalizes with pre-existing endogenous Mediator condensates. (C) Endogenous Mediator complex subunit 19 is labelled with HaloTag and shows transcriptional condensates (Cyan). (D) IDR of BRD4 fused with mCherry for visualizing shows clusters of IDR in nucleus (Red). (E) Overlay of Mediator and IDR of BRD4 confirms that Mediator condensates and IDR clusters are colocalized. (F) Intensity profile through the line of an orange arrow shown in panel E. It shows that IDR of BRD4 at a condensate site is more accumulated than Med19, compared to the background level. (H-K) NELFA colocalizes with pre-existing endogenous Pol II condensates. (G) Pol II forms endogenous condensates (Cyan). (H) NELFA fused with mCherry forms clusters of NELFA in nucleus (Red). (I) Overlay of Pol II and NELFA confirms that Pol II condensates and NELFA clusters are colocalized. (J) Intensity profile through the line of an orange arrow shown in panel I. Panel B and C were originally produced from pondr.com

FIG. 2 Colocalized IDRs are highly mobile. (A) A representative cell for a FRAP (Fluorescence Recovery after Photobleaching) experiment. At 0 sec, one condensate is bleached (Yellow box) but another condensate is not bleached (Blue box). (B) Averaged intensity profile after bleaching over time shows that IDRs of BRD4 at condensates is highly mobile. The recovery fraction of IDRs of BRD4 is 90% and the half-recovery time is around 3.4 sec. The inset shows general photobleaching of IDRs of BRD4 at condensates over time.

FIG. 3 (A) iLID (improved light-inducible dimer) and its partner sspB bind together reversibly under blue light. The dissociation constant of iLID and sspB is 130 nM under blue light and 4700 nM in dark. iLID and sspB colocalize under blue light within seconds and dissociate within minutes in dark. (B) Multivalent interaction between scFv (single-chain variable fragment) and SunTag, which has 24 binding sites for scFv, is usually utilized to enhance the signal-to-noise ratio for fluorescence imaging. The dissociation constant of scFv and SunTag is 40 pM. (C) A multivalent opto-genetic system using three components. IDR-mCh is fused to sspB (IDR-mCh-sspB) for interaction with iLID under blue light. Due to the DR part, this component stays at endogenous transcriptional condensates as shown in FIG. 1D and H. scFv and iLID are fused with sfGFP (scFv-sfGFP-iLID). 24× SunTag is adopted for efficient dragging of a special protein into condensates and better visualization of binding between iLID and sspB under blue light. 24× SunTag and scFv-sfGFP-iLID will form a complex protein containing up to 24 iLIDs. Under blue light, the complex starts to interact with IDR-mCh-sspB in nucleus and at condensates. The inventors hypothesize that because of multiple iLIDs, the complex may find a condensate quicker and have a higher binding affinity to the condensate.

FIG. 4. Light-induced targeting of endogenous condensates (LiTEC) system can accumulate GFPs into condensates upon blue light. (A-F) Cells expressing the components (IDR-mCh-sspB, scFv-sfGFP-iLID and 24× SunTag) show light-inducible targeting and accumulation of GFPs into endogenous condensates. (A) IDRs of BRD4 are in endogenous condensates. Three representative condensates are marked in yellow boxes. (B) GFP is uniformly distributed before turning on blue light. (C-E) At endogenous condensate locations, GFP accumulations are detected 5 sec, 10 sec, and 30 sec after blue light, respectively. The longer the blue light turns on, the brighter the GFP accumulations in yellow boxes are. (F) GFP intensity profile over time shows that GFPs start to accumulate upon blue light. Intensity is normalized to the background intensity in nucleus. The blue light was on at time t=0. The average intensity at three condensate locations (yellow boxes) increases gradually upon blue light but the intensity at the reference location decreases due to photobleaching. (G) When DR is not expressed in cells, scFv-sfGFP-iLID is uniformly distributed through nucleus. (H) A proposed structure for cargo delivery into condensates. Cargo should be a protein of our choice that is able to give information about the nature of condensates by manipulating functions or compositions of the condensates.

FIG. 5 LiTEC enhances IDR accumulation at endogenous condensates upon blue light (BL). (A) Representative image of BRD4 IDR before blue light exposure. (B) Image of BRD4 IDR after 50 sec of blue light exposure. The IDR clusters become bigger and brighter than clusters before blue light. (C) Intensity profile of BRD4 IDR through the orange arrow shown in panel B. Upon BL, mCh intensity at condensate locations increases. I₀ₛ is the peak mCh intensity of an IDR cluster before BL. I₅₀ₛ is the peak mCh intensity of the IDR after BL 50s. I_{B} is the background level, set to 1. FCBL5s is defined as the ratio of I₅₀ₛ−I_{B} to I₀ₛ−I_{B}. mCh signal is normalized by the mean mCh intensity of the nucleus. (D) Image of scFv-sfGFP-iLID after 50 sec of blue light exposure. One representative cluster is marked in yellow box. (E) GFP intensity in the yellow box under BL is measured over time. GFP signal is normalized by the mean GFP intensity of the nucleus at each time. Black dots are measured GFP signals over time and red line is the one-phase association curve fitted to black dots. (F) GFP relative intensity differences and mCh relative intensity differences (I₃₀ₛ−I₀ₛ) show a positive correlation (n=218). It indicates that GFP accumulation upon BL drags outside IDR into condensates. (G) Schematics of effect on compositional change caused by IDR increase upon BL.

FIG. 6 Endogenous BRD4 is not affected by BRD4 IDR enhancement upon 70 sec blue light (BL). (A) Procedure of Blue-On experiment. BRD4 IDR is firstly imaged for 5 sec to measure mCh intensity before BL (‘Before BL’). Then, GFP and Halo-BRD4 are simultaneously imaged using 488+642 nm laser and a dual camera for 70 sec. Finally, BRD4 IDR is imaged for 5 sec to measure mCh intensity after BL (‘After BL’). (B) For the marked condensate (orange arrow), Fold-Change_{mCh,BL70s} is 1.70 and Fold-Change_{BRD4,BL70s} is 0.77. BRD4 relative intensity over time, GFP relative intensity over time, and mCh relative intensity profile around the condensate are shown. (C) Procedure of Blue-Off experiment. BRD4 IDR is measured for 5 sec (‘Before’) followed by 70 sec measurement of Halo-BRD4 using 642 nm laser. No blue light is used. Then, BRD4 IDR is imaged again for 5 sec (‘After’). (D) For the marked condensate (orange arrow), Fold-Change_{mCh,NoBL70s} is 0.93 and Fold-Change_{BRD4,NoBL70s} is 0.78. BRD4 relative intensity over time and mCh relative intensity profile around the condensate are shown. (E) Statistical analysis of mCh relative intensity for each condition (n=50 for Blue-On, n=39 for Blue-Off). For Blue-On, median difference and mean difference between ‘After BL’ and ‘Before BL’ are 0.251 and 0.288. For Blue-Off, the differences is statistically not significant. (F) Statistical analysis of BRD4 Fold-Change₇₀ₛ of Blue-On and Blue-Off experiments. In general, BRD4 relative intensity decreases over time. By comparing the degree of decrease for each condition, the effect of IDR enhancement can be predicted. Here, BRD4 does not show any statistically meaningful differences between ‘Blue-On’ and ‘Blue-Off’. (Median difference=0.00234 and Mean difference=0.041; Median, Mean=0.8109, 0.8375 (±0.0176, SEM) for Blue-On, 0.8085, 0.7965 (±0.0128, SEM) for Blue-Off). It indicates that IDR enhancement upon blue light may not affect on the endogenous BRD4 in transcriptional condensates. For all the conditions in this experiment, 3D z-stack images were taken and projected into a 2D plane.

FIG. 7 Endogenous MED is slightly increased by BRD4 IDR enhancement upon 70 sec blue light (BL). The identical procedure from the previous figure was performed for this experiment except for measuring the endogenous MED, instead of the endogenous BRD4. (A) Endogenous MED is measured under blue light for 70 sec. Before and after BL, BRD4 IDR is imaged for 5 sec (‘Before’ and ‘After’). (B) For the marked condensate (orange arrow), Fold-Change_{mCh,BL70s} is 1.62 and Fold-Change_{MED,BL70s} is 0.68. BRD4 relative intensity over time, GFP relative intensity over time, and mCh relative intensity profile around the condensate are shown. (C) Endogenous MED is measured without blue light for 70 sec. Before and after this measurement, BRD4 IDR is imaged for 5 sec. (D) For the marked condensate (orange arrow), Fold-Change_{mCh,NoBL70s} is 1.04 and Fold-Change_{MED,NoBL70s} is 0.77. BRD4 relative intensity over time and mCh relative intensity profile around the condensate are shown. (E) Statistical analysis of mCh relative intensity for each condition (n=76 for Blue-On, n=55 for Blue-Off). For Blue-On, median difference and mean difference between ‘After’ and ‘Before’ are 0.269 and 0.315. For Blue-Off, the differences is statistically not significant. (F) Statistical analysis of MED Fold-Change₇0s of Blue-On and Blue-Off experiments. In general, MED relative intensity decreases over time. By comparing the degree of decrease for each condition, the effect of IDR enhancement can be predicted. Here, MED shows a tiny (~5%) but the statistically meaningful difference between ‘Blue-On’ and ‘Blue-Off’. (Median difference=0.0439 and Mean difference=0.0498; Median, Mean=0.8215, 0.8189 (±0.0096, SEM) for Blue-On, 0.7717, 0.7750 (±0.0141, SEM) for Blue-Off). It indicates that IDR enhancement upon blue light may recruit a small amount of endogenous MED into transcriptional condensates. For all the conditions in this experiment, 3D z-stack images were taken and projected into a 2D plane.

FIG. 8 Schematic illustration of BioID with LiTEC. (A) Principle of an exemplary non-limiting embodiment of the system of the invention (also referred to as LiTEC in this specification) with three components; scFv-sfGPF-iLID for multivalency and optogenetics, IDR-mCh-sspB for targeting condensates and optogenetics, and BioID-24×SunTag for biotinylation and multivalency. BioID-24×SunTag binds to twenty-four iLIDs thus it forms a giant protein complex (“BioID complex”, or “LiTEC Cargo”). BioID complexes are uniformly distributed in dark, but start to accumulate in condensates upon blue light. (B) Cartoon of expected LiTEC process. All three components are expressed in cells. BioID-24×SunTag and scFv-sfGFP-iLID form BioID complexes and IDR-mCh-sspB are accumulated in transcriptional condensates. When the blue light is on, the optogenetic interaction between sspB and iLID starts. The interaction induces BioID complexes dragged into condensates. When the blue light is off, the optogenetic interaction is disappeared. The bound BioID complexes now freely diffuse out of condensates.

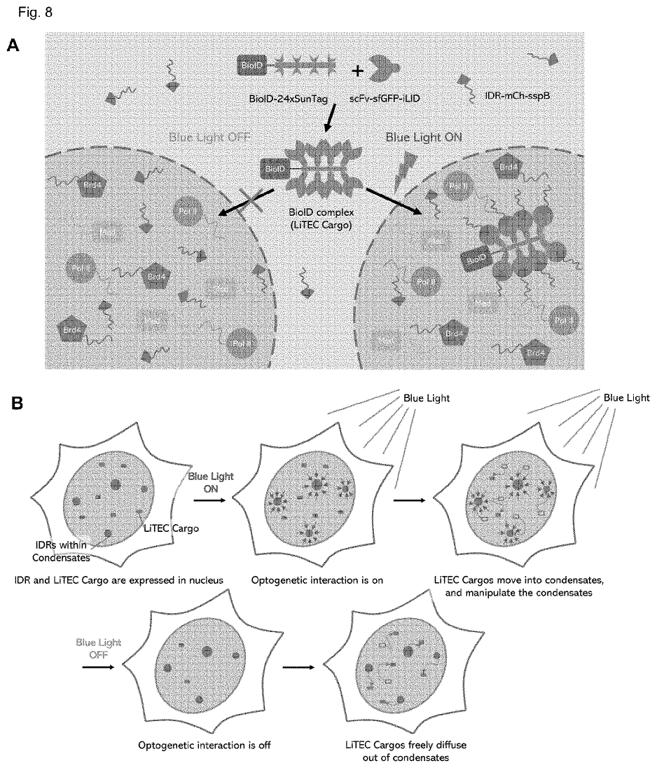

FIG. 9 Doxycycline-inducible BioID-SunTag for BioID. (A) (Left) Cell lines for BioID experiment. miniTurboID is fused to 24× SunTag (BioID-SunTag). (Right) To reduce the background level of biotinylation in nucleus, BioID-SunTag expression is controlled by a doxycycline-inducible system. (B) The amount of GFP accumulation after 30 sec of blue light exposure is dependent on doxycycline. (Top) (Left) Examples of GFP accumulation fitting over time using a one-phase association curve. Blue light turns on for 30 sec. (Middle) GFP accumulation fitted lines from No-SunTag cell lines. (Right) GFP accumulation fitted lines from the Dox-inducible SunTag cell line without Dox (Dox 0). (Bottom) GFP accumulation fitted lines from the Dox-inducible SunTag cell line with Dox 1 (Left), 2 (Middle), and 4 (Right) μg/ml for 48 hours, respectively. It indirectly indicates BioID-SunTag is successfully induced by adding doxycycline. (C) Average GFP relative intensity difference for each condition between before and after 30 sec of blue light. From left to right, mean is 0.206 (±0.144, SD and n=39), 0.315 (±0.158, SD and n=39), 2.382 (±1.135, SD and n=43).

FIG. 10 Biotinylated proteins are particularly accumulated in transcriptional condensates upon blue light. (A-D) Cartoons of expected IDR clusters, GFP accumulations, and protein biotinylation in different conditions. (A) For ‘All’ condition, GFP accumulates into transcriptional condensates (which are marked with BRD4 IDR) upon blue light, resulting in biotinylation of the condensates by BioID. Biotinylation is shown as purple colours. (B) Without blue light, BioID is not dragged into condensates, resulting in no biotinylation at condensate sites. For ‘All’ and ‘Cont1’ conditions, free BioID can biotinylate the nuclear proteins, thus this background level is shown as deeper purple than the background level in ‘cont2’ and ‘cont3’ conditions. (C) Without external biotins, miniTurboID does not work properly. Even though GFP is accumulated in condensates by blue light, accumulated miniTurboID does not biotinylate proteins in condensates. (D) Without Doxycycline treatment, BioID-SunTag complexes are not expressed, resulting in no biotinylation overall. (E-H) Immunofluorescence (IF) of biotinylated proteins using Streptavidin-Alexa647 in different conditions. It confirms the expected results from panel A to D are correct. Arrows indicate the locations of transcriptional condensates. (I and J) Statistical analysis of GFP relative intensity and Streptavidin-Alexa647 relative intensity for each condition, respectively. Only ‘All’ shows accumulated biotinylation in condensates. The middle line in the box indicates the median value and the plus mark (+) indicates the mean value. (n=36 for ‘All’, n=23 for ‘Cont1’, n=22 for ‘Cont2’, and n=21 for ‘Cont3’.)

FIG. 11 Schematics of sample preparation steps for mass spectrometry using streptavidin-biotin pull-down. Firstly, nuclear proteins are extracted after proper treatments, such as blue light exposure, biotin incubation, and doxycycline incubation. The extract contains biotinylated and non-biotinylated proteins. Since streptavidin and biotin have a very strong affinity (Dissociation constant is ~1×10⁻¹⁴ M), the biotinylated proteins are pulled down using streptavidin beads. In order to consider the non-specific binding proteins from the beads, the control bead (Streptavidin beads blocked by free biotin) experiment should be done together.

FIG. 12 Immunofluorescence for CCNT1, CDK7, and CDK9 validates the result of mass spectrometry. Primary antibodies for CCNT1, CDK7, and CDK9 are incubated followed by incubation of secondary antibody with Alexa647 for visualization. (A) Transcriptional condensates are identified by BRD4 IDR with mCh (Green color). (B) CCNT1 is visualized by indirect immunofluorescence. CCNT1 forms foci in nucleus. (C) Images of BRD4 IDR and CCNT1 are overlaid. Orange arrows mark colocalized foci and sky-blue arrows mark non-colocalized IDR-only foci. It shows that most transcriptional condensates colocalize with CCNT1 clusters. (D) The red box in panel C is magnified. Scale bars, 1 μm. (E) Relative intensity profiles through the arrow line in panel D are shown. It confirms that transcriptional condensates and Cyclin T1 clusters are colocalized. (F-H) CDK7 is labelled by indirect immunofluorescence and transcriptional condensates are marked by IDR. CDK7 forms a lot of foci but the CDK7 foci are not colocalized with transcriptional condensates. (I) The red box in panel H is magnified. Scale bars, 1 μm. (J) Relative intensity profiles through the arrow line in panel I are shown. It confirms that transcriptional condensates and CDK7 clusters are not colocalized. (K-M) CDK9 is visualized by indirect immunofluorescence. CDK9 forms foci in nucleus and some of them are colocalized with transcriptional condensates. (N) The red box in panel M is magnified. Scale bars, 1 μm. (O) Relative intensity profiles through the arrow line in panel N are shown. It confirms that only relatively small transcriptional condensates are colocalized with CDK9 clusters.

### EXAMPLE: DEVELOPING A SYSTEM FOR LIGHT-INDUCED TARGETING OF ENDOGENOUS CONDENSATES

### Example 1: Targeting Endogenous Transcriptional Condensates

In order to study the nature of biomolecular condensates, new methods are needed that preserve an intact environment inside and outside the condensates during the experiment, within the cell. Isolating intact biomolecular condensates from cells is possible. But isolating a condensate disturbs its surroundings, and especially, isolation of the liquid-like and small-sized (<1 μm) biomolecular condensates is extremely challenging. The isolated P-granules and nucleoli in previous studies were relatively large (>1 μm). Thus, in order to study transcriptional condensates, which are liquid-like and usually less than 1 μm, the inventors set out to develop a new in vivo system that is able to target and manipulate endogenous transcriptional condensates.

### Zip-Code for Condensates; Intrinsically Disordered Region (IDR)

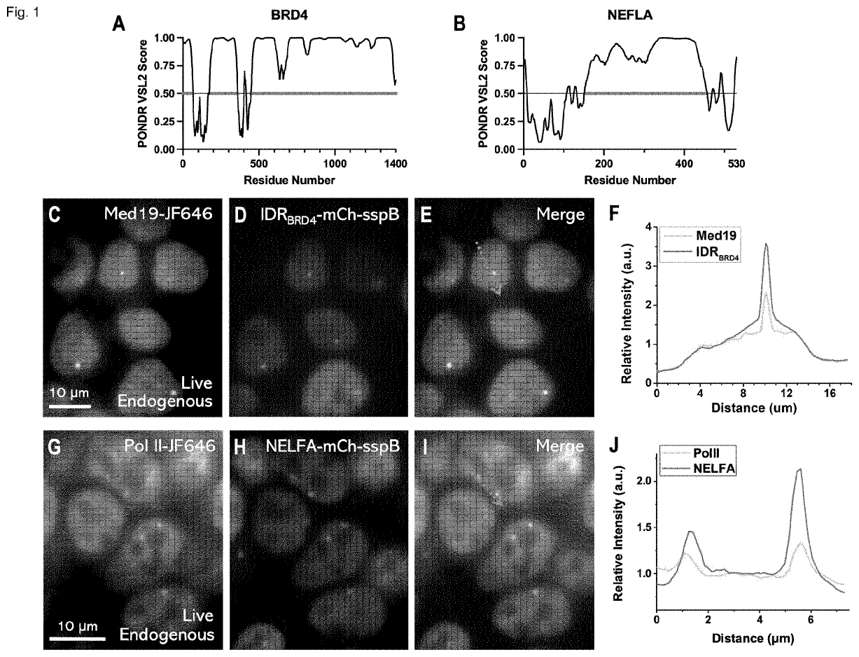

Formation of biomolecular condensates results from thermodynamic interactions between different biomolecules. Many studies have shown that weak and multivalent interactions such as ionic interactions from intrinsically disordered regions are promoting phase separation (FIG. 1). The inventors hypothesized that IDR having the same grammar as transcriptional condensates may preferentially locate in the transcriptional condensates so that over-expression of these IDR can be used to target the condensates. Unlike structured regions such as DNA-binding domains, IDR are thought to not have a specific function except weak interactions with other proteins. Thus, using IDRs seemed a reasonable approach as a lack of binding ability may minimize possible functional or compositional changes in condensates upon the over-expression of IDRs in living cells.

The inventors chose two natural IDRs from transcription-related proteins, BRD4 and NELFA, to test whether they are indeed able to colocalize with the transcriptional condensates. BRD4 is a transcription factor that is enriched in super-enhancer regions and binds to acetylated histones to up-regulate the transcription of genes. NELF (Negative elongation factor) is a four-subunit protein complex and down-regulates transcription by pausing RNA Pol II. The NELFA subunit was singled out for testing. Intentionally, the inventors selected functionally opposite proteins to demonstrate the concept of IDR targeting does not depend on protein function but on structural similarity of IDRs forming the condensate.

The inventors used a website-based amino acid sequence analysis algorithm, PONDR (pondr.com), to identify intrinsically disordered regions of BRD4 and NELFA protein. FIG. 1A and B show the PONDR VSL2 score of NRD4 and NELFA. X-axis is amino acid (aa) positions and Y-axis is VSL2 score, which shows how the amino acid sequences are significantly disordered (the regions with high VSL2 scores are intrinsically disordered regions). IDRs are marked as black bold lines at the middle of the y-axis. For BRD4, only the IDR part of the BRD4 sequence was used, from position 462 to the C terminal end of the BRD4 sequence (1362). For NELFA, since this protein's IDR is at the middle of NEFLA, the whole NEFLA protein was used for testing. In order to express them in mESCs, two plasmids were generated by standard recombinant methods; one contains a sequence of BRD4 IDR and mCherry (mCh), and the other has the whole NELFA sequence and mCh sequence. mCh is used for imaging BRD4 IDR and NELFA. Then, DNA sequences are integrated into an mESC genome by lentiviral transduction.

Endogenous Mediator or Pol II condensates are imaged in living mESC using an epifluorescence microscope (FIG. 1C and G, respectively). CRISPR/Cas9 genome edition system was used to add HaloTag sequence to the N-terminal of Rpb1 (a subunit of Pol II) and Med19 (a subunit of Mediator), then Halo-JF646 was added for imaging. Then, BRD4 IDR and NELFA are imaged as FIG. 1D and H. Both BRD4 IDR and NELFA show clusters in the nucleus, and overlay with endogenous Mediator or Pol II image confirms their clusters are colocalized with Mediator condensates or Pol II condensates (Fig. E and I, respectively). The intensity profile of BRD4 IDR and NELFA through an orange arrow in Panel E and I are shown in Fig. F and J, respectively. It shows that both BRD4 IDR and NELFA have a higher condensate-to-background intensity ratio than that of Mediator or Pol II. Overall, this result confirms that BRD4 IDR and NELFA can be used to target the endogenous transcriptional condensates in living mESCs.

Colocalization of BRD4 IDR to transcriptional condensates confirmed with expectations because of its functional relation to super-enhancers and transcription, but colocalization of NELFA is an unexpected result because transcriptional condensates are speculated to up-regulate transcription due to concentrated transcription apparatus. NELF, however, is a transcription down-regulator by pausing Pol II but it may have a similar biomolecular grammar with other transcription factors. It reflects the fact that the functions of transcriptional condensates is not fully understood. One way to refer to the IDRs that can target a specific biomolecular condensate is as “zip-codes” for the biomolecular condensate. Different IDR target different types of biomolecular condensates, which enables to target the specific condensates by changing “zip-code” of IDRs.

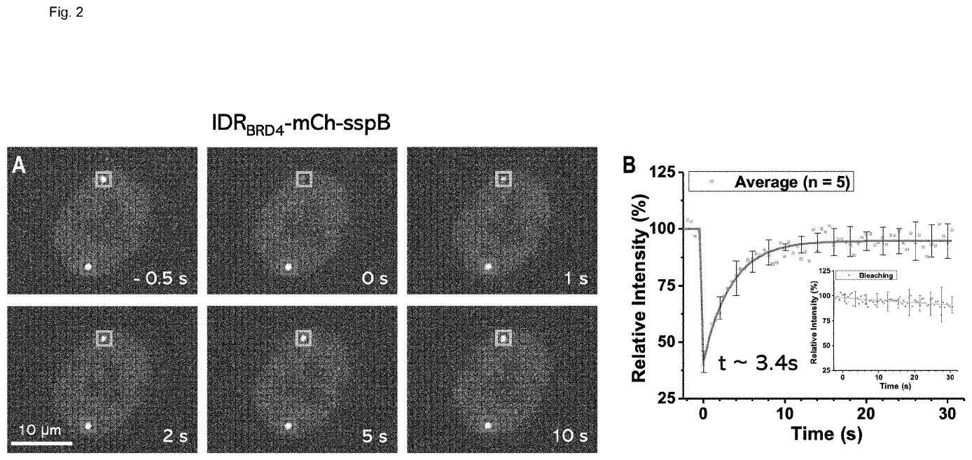

FRAP experiments were performed on BRD4 IDR clusters to test their mobility. FIG. 2A shows the time-lapse images of a representative cell. Two BRD4 IDR clusters are marked as yellow box (top) and blue box (bottom), and a cluster in yellow box is bleached at time t=0 but a cluster in blue box is control. At t=0, the cluster is bleached so that the intensity level is similar to the signal in nucleus. But after 1 sec of bleaching, it immediately starts to recover. Panel B shows the averaged intensity profile of BRD4 IDR clusters after bleaching as a function of time (n=5). It shows that BRD4 IDR recovers rapidly. Its half-recovery time is 3.4 sec, and the recovery fraction is 90%. Inset in panel B is an intensity profile of the control cluster in blue box. Due to photobleaching, it monotonically decreases.

In summary, the IDR part of two proteins, BRD4 and NELF was assayed for its ability to target the transcriptional condensates of Mediator and Pol II in mESCs. It was confirmed that both BRD4 IDR and NELFA form clusters in mESCs and the clusters are colocalized with transcriptional condensates. According to intensity analysis, even their condensate-to-background ratio is higher than that of Mediator or Pol II. BRD4 DR is also highly mobile. Thus, by using BRD4 IDR, the inventors successfully targeted the endogenous transcriptional condensates. The first condition for a new system is confirmed.

### Optogenetics and Multivalency in an Exemplary Embodiment of the System

After establishing that targeting of transcriptional condensates using appropriate zip codes (IDRs) is possible, the next step was to find a way to manipulate the transcriptional condensates temporally but effectively. Here, temporal manipulation is important. If manipulation of the condensates is effected over a temporal continuum, it might not be possible to distinguish whether the features of the condensates obtained from the manipulation are solely attributed to the natural properties of condensates or to influence the artificial manipulation of condensates.

Optogenetics is a biological technique that employs light to control the activities of cells, tissues, or even organs. The concept of optogenetics came from neuroscientific research on channel rhodopsin, which is a light-gated ion channel. Many groups have used optogenetic tools in various fields for precise and simultaneous response controls. In the biomolecular condensate field, studies have used optogenetics to make artificial biomolecular condensates (see Bracha et al., (2018) Cell, 175(6):1467-1480.e13; Shin et al. (2017) Cell, 168(1-2):159-171.e14; Shin et al. (2018) Cell, 175(6):1481-1491.e13; Shunsuke et al. (2021) Nature, 599(7885):503-506). Here, instead of making artificial condensates, the inventors adopted optogenetic components used by these authors to target and drag proteins of interest into pre-existing endogenous transcriptional condensates upon blue light.

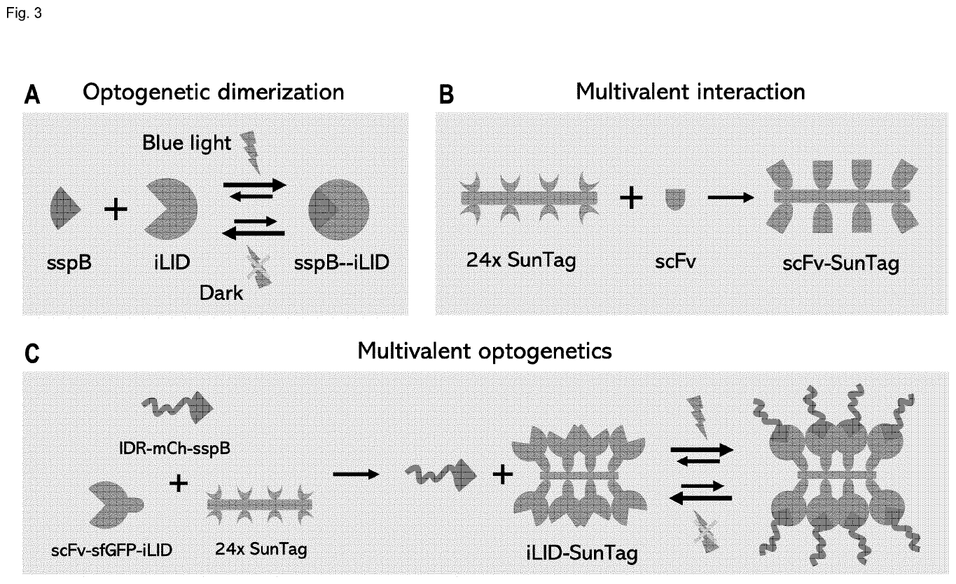

LID (improved light-inducible dimer) and sspB, one of the optogenetic systems, are introduced in FIG. 3A. LID changes its conformation when it is exposed to blue light (450 to 500 nm) so that the binding affinity to sspB increases dramatically.

The dissociation constant Ko of LID and sspB is 4700 nM in dark but becomes 130 nM under blue light, thus their binding under blue light is 36 times stronger than their binding in dark. Also, their binding process is reversible. They start to bind in seconds upon blue light and dissociate in minutes when blue light is off, offering control of the signal instantly by modulating the intensity of blue light.

Additionally, the SunTag system was used to drag proteins of interest more efficiently and also for easier imaging due to its high signal-to-noise ratio. Fig. B shows SunTag and scFv (single chain variable fragment). 24× SunTag has 24 peptide epitopes to which scFv can bind, thus it provides multivalent interaction. The dissociation constant K_{D} of scFv and SunTag is 40 pM. SunTag is commonly used to enhance the fluorescence signal-to-noise ratio (SNR).

The design of one exemplary non-limiting embodiment of the system according to the invention is shown in FIG. 3C. It contains three components; BRD4 DR is fused with mCh and sspB (IDR-mCh-sspB), LID is fused with scFv and sfGFP (scFv-sfGFP-iLID), and 24× SunTag. Basically, scFv and SunTag bind all the time, it will form a complex containing up to 24 sfGFPs and iLIDs. Then, upon blue light exposure, this complex starts to bind to up to 24 IDR-mCh-sspB. Shin et al used similar components to make artificial droplets in HEK293mcells or U2OS cells and called this system CasDrop (due to dCas9 being fused to SunTag), but in the present work, these three components will be used to drag proteins of interest into pre-existing endogenous condensates. This is attributed to IDRs staying in the endogenous condensates in mESCs. iLID-SunTag complexes will come into the endogenous condensates in order to interact with sspB, which is fused to IDR. The inventors refer to this three-component system by the acronym “LiTEC”, for “light-induced targeting of endogenous condensates”.

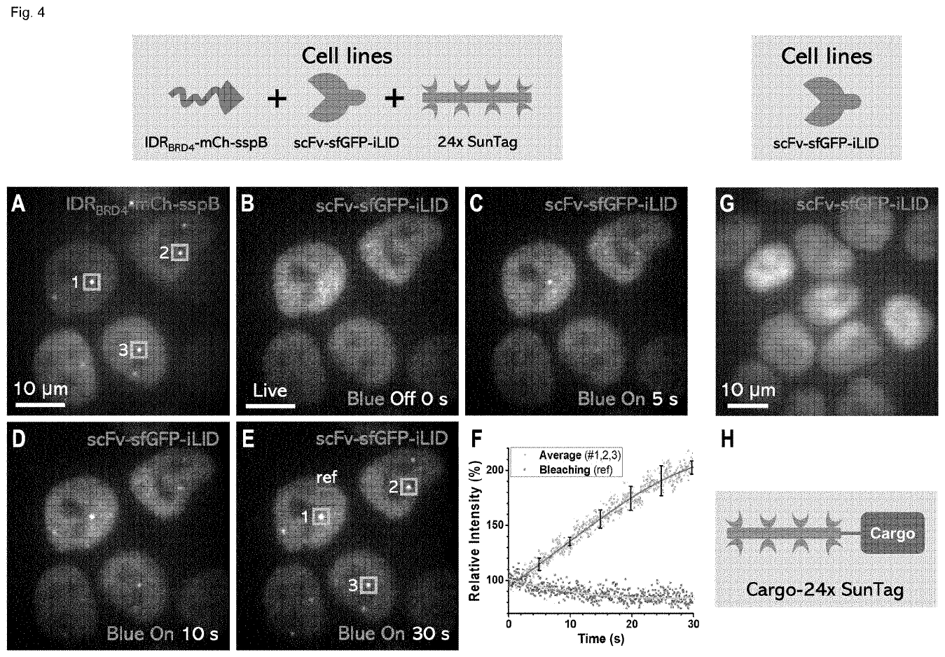

In order to test whether LiTEC is indeed able to drag proteins that were originally distributed uniformly in nucleus, into pre-existing endogenous condensates upon blue light, localization of GFP labelled proteins is observed. All three components were expressed in mESCs. IDR-mCh-sspB is imaged by 561 nm laser (FIG. 4A). As we have already checked, BRD4 IDR is accumulated in endogenous transcriptional condensates. Three representative clusters are highlighted in yellow boxes. Then, by 488 nm laser, scFv-sfGFP-iLID is imaged. Since 488 nm laser itself is blue light, imaging this component immediately induces optogenetic binding. But LID and sspB still need a few seconds to interact, thus, at the moment of blue light turn-on (time t=0), GFP signals are (yet) uniformly distributed in nucleus (FIG. 4B). In the case of a cell line containing only scFv-sfGFP-iLID component, cells show only uniform distribution of GFP even the cells are under blue light. This is because LID does not have its binding partner, sspB (FIG. 4G). However, in LiTEC, GFPs start to accumulate into DR clusters after a few seconds of blue light (FIG. 4C-E). Averaged GFP intensity at three representative DR clusters is shown as a function of time in FIG. 4F. It increases rapidly over time but GFP intensity at reference monotonically decreases due to photobleaching. This result indeed confirms that iLID-SunTag complexes are successfully inserted into the pre-existing transcriptional condensates upon blue light.

This experiment however demonstrates the insertion of proteins devoid of enzymatic activity (GFP and SunTag) into the condensates. The inventors wondered if it was possible to insert proteins with enzymatic function to manipulate the condensates or to effect transcription? Their assumption was that a protein (cargo) fused with 24× SunTag might be transported into the condensates when the SunTag is fused with DR upon irradiation with blue light (FIG. 4H). In principle, any cargo can be transported.

But before exploring this model further, the inventors analyzed the biophysical effects of LiTEC on transcriptional condensates. This system artificially inserts some exogenous components which do not exist naturally in the condensates so they may affect the nature of transcriptional condensates. In the next subsection, they show results of experiments aimed at testing how the LiTEC system affects the composition of transcriptional condensates.

### Quantitative Compositional Change Analysis During LiTEC

The hypothesis underlying the work performed here was that some BRD4 IDRs positioned outside of condensates (‘outside IDR’) originally may come into condensates upon blue light due to the multivalency of iLID-SunTag complex. iLID-SunTag complex would bind more likely to ‘outside IDRs’ first before finding ‘inside IDRs’, which are in the condensates. But once one of LID in iLID-SunTag complex binds to ‘inside IDRs’, other iLIDs in iLID-SunTag complex would easily find IDRs for binding due to the high concentration of IDR in condensates. Thus, the iLID-SunTag complex will stay in the condensates. During this process, ‘outside IDRs’ may come into the condensates with the iLID-SunTag complex, resulting in a higher intensity of IDR in the condensates compared to the intensity before blue light.

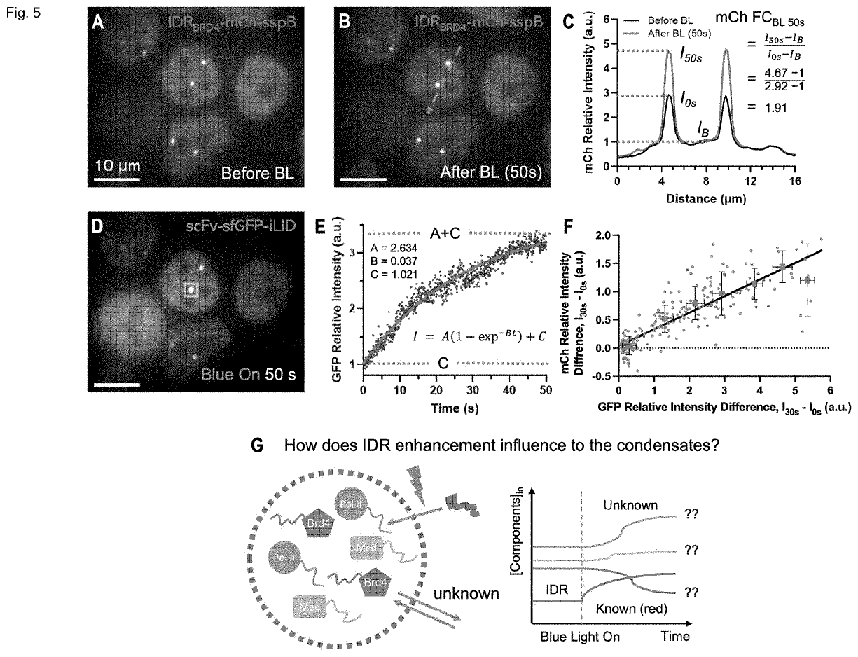

In order to test this hypothesis, IDR-mCh-sspB were imaged without blue light using 561 nm laser for a few seconds. Then, imaging scFv-sfGFP-iLID with blue light (488 nm laser) for 1 min. Finally, IDR-mCh-sspB were imaged again without blue light for a few seconds. Ideally, the variation of mCh signals over time under blue light would be observed, which is not possible due to crosstalk between mCh and GFP. FIG. 5A, B show the distribution of IDRs in nucleus before blue light exposure (‘Before BL’) and after 50 sec blue light exposure (‘After BL (50s)’), respectively. FIG. 5D and E show GFP distribution in nucleus at the moment of 50 sec blue light exposure and GFP relative intensity profile over time under blue light, respectively. Here, ‘relative intensity’ is the maximum intensity at condensate divided by the mean intensity in the nucleus' background.

$$Relative intensity = Maximum intensity at condensate / Mean intensity in nucleus$$

If a relative intensity is equal to 1, it means the intensity at a condensate site is equal to the intensity of the background. If the background level is to be set to zero, a similar term, ‘relative change’ can be used.

$$Relative change = ( Intensity at condensate - Background intensity ) / Background intensity$$

The relative change is the true signal out of the background, so it is similar to the concept of signal-to-noise ratio, but with normalization (Background is 1). If the difference of relative intensities at different time points are to be determined, one can calculate a relative intensity difference:

$$Relative intensity difference = Relative change at Time A - Relative change at Time B = Relative intensity at Time A - Relative intensity at Time B$$

Another term, ‘Component Fold-Change(FC)_{BL/NoBL,Time}’, is introduced as a ratio of the relative change of a component after the time with/without blue light (BL) to the initial relative change of the component. Two examples illustrate this concept:

$${Ratio}_{mCh , BL 60 s} = \frac{mCh relative intensity measured after 60 s of blue light}{mCh relative intensity measured before blue light exposure}$$

$${Ratio}_{BRD 4 , NoBL 60 s} = \frac{BRD 4 relative intensity measured after 60 s without BL}{BRD 4 initial relative intensity}$$

The intensities of BRD4 IDR and LID can be compared before and after blue light exposure. FIG. 5C shows BRD4 IDR intensity profile through the orange arrow shown in panel B. The black line represents the initial relative intensity of BRD4 IDR. The red line represents the relative intensity of BRD4 IDR after 50 s of blue light. At the first condensate, the initial relative intensity is marked as Ios and the relative intensity after 50 s of blue light is marked as I₅₀ₛ. Then, the fold-change of this condensate, mCh Fold-ChangeBL50s, is (I50s−IB)/(I0s−IB). For this representative condensate, Fold-ChangeBL50s=1.91. It confirms that BRD4 IDRs are additionally accumulated into transcriptional condensates upon blue light. More specifically, after 50 sec of blue light, mCh relative change has 91% higher relative changes than its initial relative change. Thus, calculating ‘Fold-Change’ is a good way to compare initial and final relative change based on the initial relative change2. Like mCh signals, the relative intensity of GFP signal as a function of time is presented in FIG. 5E. Again, GFP accumulation at condensates during blue light exposure is confirmed. The GFP accumulation at condensates over time can be interpreted as association kinetics of LID and sspB, thus GFP relative intensity over time can be fitted by the one-phase association equation

$$I ( t ) = A ( 1 - {exp}^{( - Bt )} ) + C$$

Finally, the correlation between GFP accumulation and IDR enhancement is tested by drawing a scatter plot of ‘GFP relative intensity difference’ and ‘mCh relative intensity difference’ (Relative intensity_{BL30s}−Relative intensityBLos). For this experiment, mCh relative intensities were measured before and after blue light and GFP relative intensities before and after 30 s of blue light. FIG. 5F shows that GFP relative intensity differences and mCh relative intensity differences are positively correlated. In other words, the more GFPs are accumulated into condensates upon blue light, the more ‘outside IDRs’ are also accumulated into condensates. How does IDR accumulation affect to the nature of transcriptional condensates? Not like SunTag, multivalency of BRD4 IDRs may make them possible to interact with other components so it might have an impact on the composition of condensates. FIG. 5G briefly describes the unknown effects of IDR enhancement upon blue light. While the blue light is on, IDR concentration in the condensates starts to increase, and it might increase or decrease or does not change the concentration of endogenous components such as BRD4, MED, and Pol II. To test how the endogenous proteins react to IDR enhancement by labelling them, the endogenous BRD4 were labelled using HaloTag to elucidate how BRD4 condensate reacts upon an increment of BRD4 IDR. Two experiments were designed:

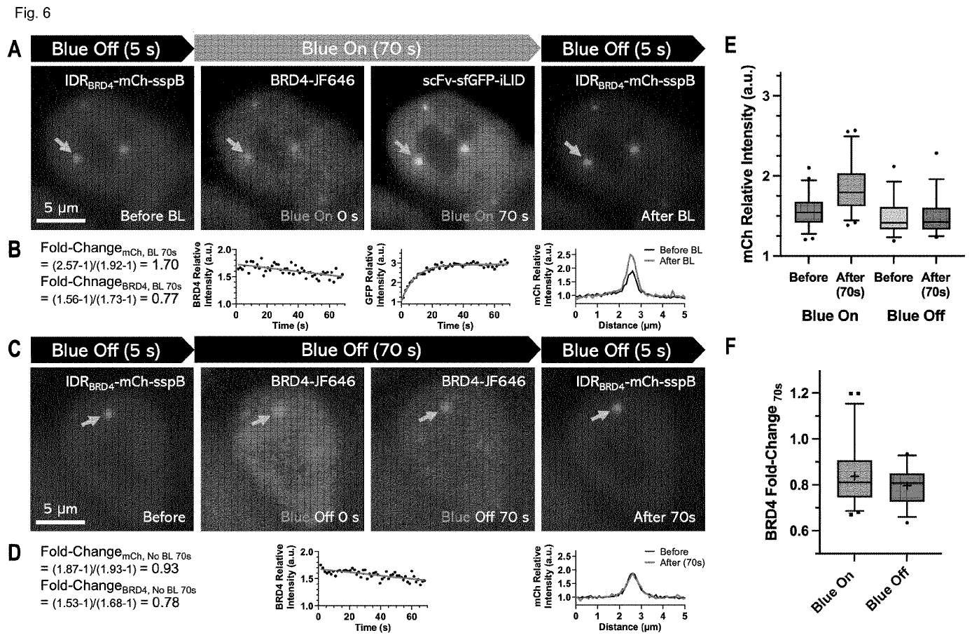

A) Turning on blue light for a minute and watching GFP signal change, additionally watching the endogenous BRD4 while blue light is on (‘Blue-On’ experiment). FIG. 6A describes how the first experiment proceeds. Before blue light is on (‘Before BL’), BRD4 IDR is firstly imaged for 5 sec by imaging mCh signals with 561 nm laser. Then, by using 488 and 642 nm lasers and a dual camera, GFP signals and Halo-BRD4 signals are simultaneously imaged for 70 sec. Before performing this imaging, 100 nM of Halo-JF646 dye is added for 15 min and washed with 2i media twice. For all images, 11 slices of z-stack are taken with 300 nm gap so that condensates of interest are not out of focus during acquisition. After 70 sec of blue light (‘After BL’), BRD4 IDR is imaged again for 5 sec to check how much it increases by 70 sec of blue light. In FIG. 6B (endogenous) BRD4 relative intensity and GFP relative intensity are plotted as a function of time. In general, BRD4 signals decrease due to bleaching, and GFP signals reach their peak within a minute. Fold-Change_{BRD4,BL70s} is 0.77 for the pointed condensate (orange arrow). It means that the BRD4 relative change after 70 sec of blue light decreases 23% from its initial relative change. mCh relative change before and after blue light around the pointed condensate also shows that IDR relative change increases 70% compared to its initial relative change. (Fold-Change_{mCh,BL70s} is 1.70 for this condensate.) However, only with this experiment, one cannot conclude whether BRD4 fold-change decrease is due to IDR enhancement, or photobleaching. The control experiment is described in FIG. 6C. Halo-BRD4 is imaged for 70 sec without blue light exposure (‘Blue-Off’ experiment). Before and after BRD4 imaging, BRD4 IDR is also imaged for 5 sec (‘Before’ and ‘After’). Thus, GFP signals are not measured. BRD4 relative intensity profile over time and mCh relative intensity profile through the pointed condensate are shown in FIG. 4-6D. BRD4 signals decrease over time and mCh signal does not change much. For this case, Fold-Change_{BRD4,NoBL70s} is 0.78 and Ratio_{mCh,BL70s} is 0.93.

To arrive at a meaningful conclusion, two groups of Halo-BRD4 signal fold-change were compared statistically. 50 condensates for ‘Blue-On’ and 39 condensates for ‘Blue-Off’ are analyzed. When blue light is off (‘Blue-Off’), mCh relative intensity before and after 70 sec of BRD4 imaging does not show difference. However, with blue light (‘Blue-On’), mCh relative intensity increases; median and mean difference between ‘After BL’ and ‘Before BL’ is 0.251 and 0.288, respectively (FIG. 6E). Median and mean value of mCh fold-change (After BL to Before BL) in ‘Blue-On’ condition are 1.548 and 1.537, and that in ‘Blue-Off’ condition are 1.000 and 1.021 (Data not shown). Finally, BRD4 fold-change in ‘Blue-On’ condition and ‘Blue-Off’ condition is analyzed in FIG. 4-6F. Median and mean value are 0.8109, 0.8375 (±0.0176, SEM) for ‘Blue-On’ and 0.8085, 0.7965 (±0.0128, SEM) for ‘Blue-Off’, indicating that their difference is not meaningful (Numerically, median difference=0.00234 and mean difference=0.041). In other words, IDR enhancement (In this case, 54.8% increase of BRD4 IDR) does not impact on the endogenous BRD4 intensity.

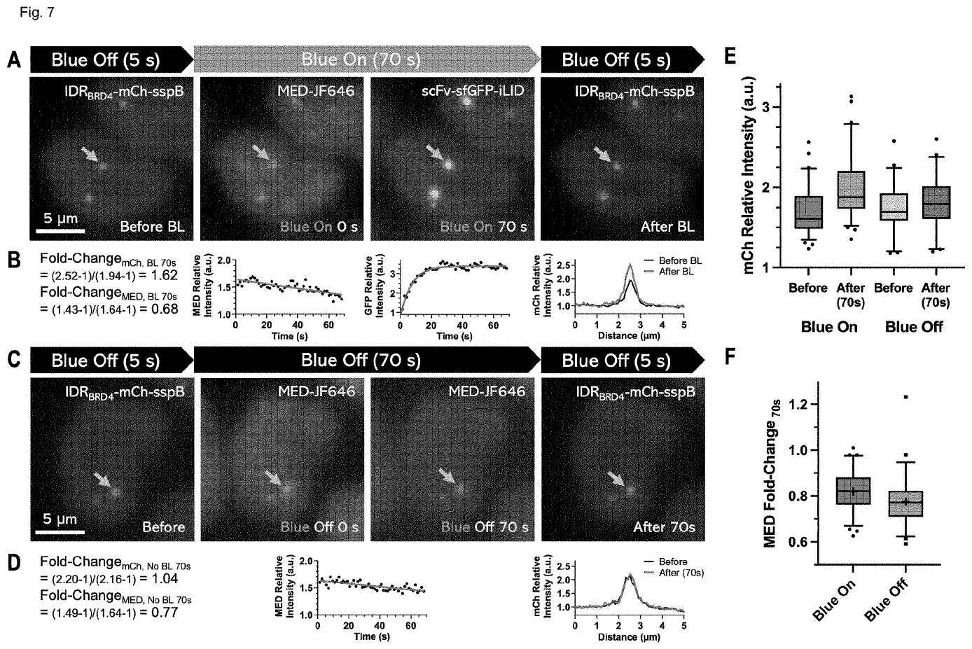

The endogenous Mediator (MED) is labeled with HaloTag and imaged by the identical protocols that were used for BRD4 experiments (FIG. 7). 76 condensates for ‘Blue-On’ and 55 condensates for ‘Blue-Off’ are analyzed. Panel E shows that mCh relative intensity changes after blue light, while it does not change without blue light. For ‘Blue-On’, median and mean difference are 0.269 and 0.315, respectively. Median and mean value of mCh fold-change (After BL to Before BL) in ‘Blue-On’ condition are 1.438 and 1.495, and that in ‘Blue-Off’ condition are 1.098 and 1.126 (Data not shown). Lastly, MED fold-change of ‘Blue-On’ and ‘Blue-Off’ is analyzed in FIG. 7F. Median and mean is 0.8215, 0.8189 (±0.0096, SEM) for ‘Blue-On’ and 0.7717, 0.7750 (±0.0141, SEM) for ‘Blue-Off’. In other words, median difference is 0.0439 and mean difference is 0.0498, which are slightly higher than median and mean difference of BRD4 fold-change from the previous experiment. Statistically, MED fold-changes from ‘Blue-On’ group and ‘Blue-Off’ group are distinguishable, even though their median or mean difference is not significant (only around 4.4% increase with blue light). It can be interpreted that there are 4.4% more endogenous MED in condensates after 70 sec of blue light, compared to the control (‘Blue-Off’) experiment. It indicates that IDR enhancement (In this case, 43.8% increase of BRD4 IDR) upon blue light might change the thermodynamics of the condensate slightly more favorable to Mediator so that the small amount of endogenous Mediator, which was originally outside of the condensate, enter into the condensate.

For both cases, mCh signals at condensates increase more than 40% after 70 sec of blue light and it results in no difference in the number of endogenous BRD4 but a slight increase in the number of endogenous MED in the condensates. The inventors' current hypothesis about these results is as follows; BRD4 binds to acetylated histones using two Bromo-domains. Since BRD4 IDR enhancement in condensates does not change the number of acetylated histones in condensates, the number of binding sites for BRD4 still remains the same. Thus, even though the number of IDR is increasing, it does not really result in the increment of endogenous BRD4 in the condensate. However, it may increase the multivalency of the condensate so that the ability to interact with other biomolecules may slightly increase from IDR enhancement. Thus, the condensates may recruit more biomolecules especially the biomolecules interacting directly with BRD4 IDR, such as Mediator. Of course, BRD4 IDR also interacts with other BRD4 but maybe IDR-IDR weak interaction is not the main factor for BRD4 recruitment. Still, it is hard to conclude firmly that IDR enhancement does not affect BRD4 but increases the number of Mediator in condensates. One can conclude that the LiTEC system does not significantly affect the nature of transcriptional condensates even though the LiTEC inserts a lot of external biomolecules. Thus, the LiTEC system reliably keeps the original nature of the condensates subject of the study.

### Example 2: Modifying Endogenous Transcriptional Condensates

The previous Example 1 introduced the possibility of the LiTEC system to study the features of transcriptional condensates. The system exemplified therein could transport iLID-SunTag complexes and subsidiary IDRs into condensates in reaction to blue light. Then, is transporting a protein of interest possible by LiTEC? The inventors hypothesize that cargoes can be inserted into condensates if they are appropriately fused IDRs, possibly multiplexed by SunTag complexes as described in FIG. 4H.

In order to test this hypothesis, proximity-based labelling methods, such as DamID and BioID, were considered. DNA adenine methyltransferase identification (DamID) is used to detect the binding sites of DNA- and chromatin-binding proteins in eukaryotes by fusing Dam (DNA adenine methyltransferase) protein. Dam proteins recognize a specific DNA sequence (GATC) nearby (~10 nm) and methylate its adenine residue. In eukaryotes, adenine methylation does not occur naturally, thus the methylated adenine regions would reflect the binding sites of the DNA-binding proteins. Biotin identification (BioID) is used to detect the interacting proteins of a protein of interest by fusing a BirA protein. BirA protein biotinylates nearby proteins within ~10 nm. Biotinylated proteins can be pulled down by streptavidin beads and be sequenced by mass spectrometry. A list of the identified proteins would reflect the list of proteins that closely interact with the protein of interest.

BioID was selected as an exemplary candidate because of the biotin tag's ability to reveal what other biomolecules are in the transcriptional condensates, and also because the biotinylated proteins are readily identified by isolation using streptavidin and subsequent MS.

### Biotinylation Identification (BioID) and LiTEC

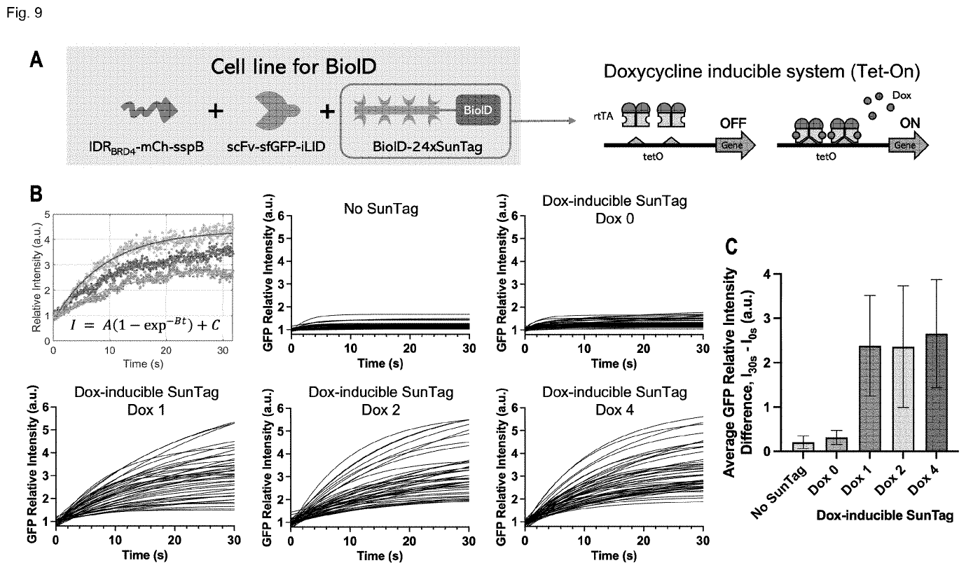

To test biotinylation by BioID using LiTEC, BioID is firstly fused to 24×SunTag to generate a cell line shown in FIG. 9A. Here, miniTurboID is chosen for BioID due to the small size, quick biotinylation speed (10 min) and comparatively low biotinylation level without exogenous biotin. Even though the level of biotinylation from miniTurboID without external biotin addition is low, the inventors intended to minimize the non-specific biotinylation happening during cell culture. Thus, they adopted the Doxycycline(Dox)-inducible system to control the expression level of BioID-SunTag complex. Doxycycline-inducible system uses tetO (tetracycline operator) sequences and rtTA (reverse tetracycline-controlled trans-activator (Gossen et al., Science, 268(5218):1766-1769, 1995; Atze, Curr. Gene Ther., 16(3):156-167, 2016). The tetO sequences are used as a promoter of the gene of interest. Only under tetracycline or doxycycline treatment, rtTA starts to bind to tetO sites and promotes transcription of the gene of interest. Throughout this experimental description, wherever the specific incubation time of Dox is not mentioned, all BioID experiments are performed after 48 hours of Dox incubation.

In order to test whether the Dox-inducible system works well and how doxycycline concentration influences the LiTEC system, cells were incubated with different Dox concentrations. As a control, a cell line only containing IDR-mCh-sspB and sfFv-sfGFP-iLID, but no SunTag expression (‘No SunTag’), is used. The Dox-inducible BioID-SunTag cell line with Dox 0, 1, 2, 4 μg/ml is incubated for 48 hours, then GFP accumulation into condensates upon blue light are imaged. FIG. 9B shows fitted GFP relative intensity over time for each condition. The first panel shows examples of raw data of GFP relative intensity over time and fitted lines (The examples are from Dox 1 condition). Fitting is done by a one-phase association curve as described in 4.6. The control experiment shows that there is a low level of GFP accumulation. Under blue light, sspB and LID binds so the small amount of GFPs interacts with sspB in condensates. Cells without Dox also show similar GFP accumulation results, indicating that the expression of BioID-SunTag complex is repressed.

With Dox 1, 2, 4 (μg/ml) treatment, GFP accumulations are highly enhanced, indicating that BioID-SunTag complexes are expressed successfully. The mean GFP relative intensity differences for each condition between before and after 30 sec of blue light are shown in FIG. 9C. For ‘No SunTag’ and ‘Dox 0’, the mean is 0.206 (±0.144, SD and n=39) and 0.315 (±0.158, SD and n=39). For ‘Dox 1’, ‘Dox 2’, and ‘Dox 4’, the mean is 2.382 (±1.135, SD and n=43), 2.362 (±1.371, SD and n=46), and 2.654 (±1.217, SD and n=51), respectively. These results confirm that the expression level of BioID-SunTag complexes is doxycycline-inducible so that we can reduce the background level of biotinylation from miniTurboID. Plus, it indirectly confirms that BioID is inserted together into condensates upon blue light.

Streptavidin conjugated with Alexa647 (Streptavidin-Alexa647) can directly visualize the biotinylated proteins. For this experiment, all cells are fixed using 4% PFA for 10 min in room temperature followed by 0.5% Triton X-100 for 5 min in room temperature. Then, cells are blocked by 2% BSA overnight. Finally, Streptavidin-Alexa647 (1:1500) is incubated in 2% BSA solution for 1 hour. Between each step, the samples are washed three times with 1×PBS.

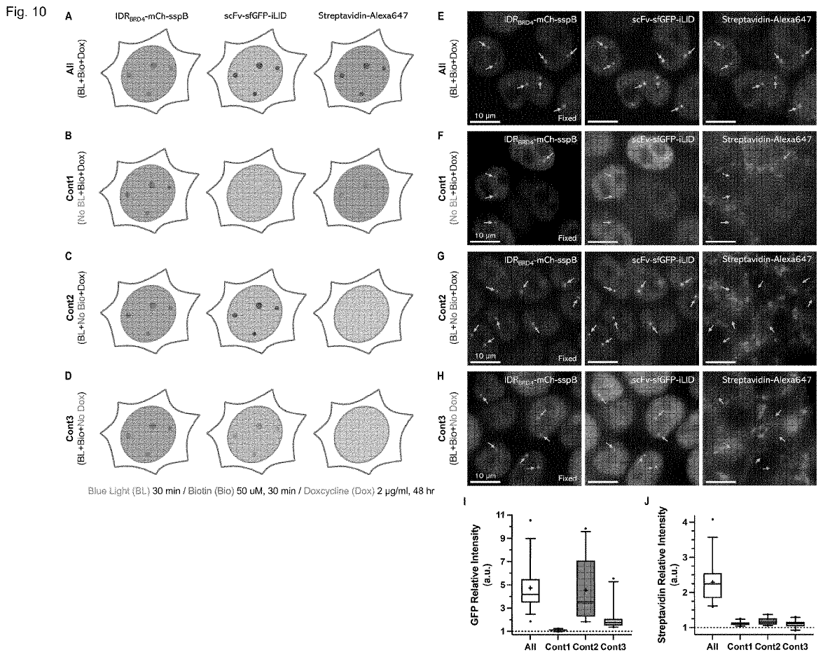

There are three variables for this experiment; Blue light, external biotin, and doxycycline. The most important four conditions to test: In ‘All’ condition, cells are treated with all three variables. Cells are firstly incubated with Dox 2 (μg/ml) for 48 hours and external biotin (50 μM) is added just before blue light is on. Blue light is on for 30 min to give enough time for biotinylation. These conditions are referred to as BL (Blue light, 30 min), Bio (Biotin 50 μM, 30 min), and Dox (Doxycycline 2 μg/ml, 48 hours). The first control (‘Cont1’) is cells treated without BL but with Bio and Dox. The second control (‘Cont2’) is cells treated without Bio but with BL and Dox. Finally, the third control (‘Cont3’) is cells treated without Dox but with BL and Bio. The expected results from these conditions are shown in FIG. 10A to D. Here, biotinylation is shown as purple color. The more concentrated biotinylation is expected, the deeper purple color is shown.

For ‘All’, it was expect to have highly accumulated biotinylations in transcriptional condensates (Deep purple dots in nucleus), but the background biotinylation level is also considerable. This is because BioID which are not dragged into condensates may biotinylate nuclear proteins randomly. For ‘Cont1’, which does not use blue light, the same background biotinylation level for ‘All’ is expected but no accumulation of biotinylation in condensates. For ‘Cont2’ and ‘Cont3’, the inventors expect a very low level of biotinylation in nucleus because cells do not have enough biotin to use (‘Cont2’) or cells do not express BioID (‘Cont3’). Of course, no accumulation of biotinylation in condensates is expected. If the expectation is correct, one may be able to get a list of “transcriptional condensate proteins” (proteins staying in transcriptional condensates) by direct comparison of a result from the condition ‘All’ and a result from the condition ‘Cont1’. Additional validation is achieved by comparing the result from condition ‘All’ to a result from condition ‘Cont2’ or ‘Cont3’.

FIG. 10E to H show BRD4 IDR, GFP, and Streptavidin-Alexa647 signals in cells under four conditions. Arrows indicate the locations of transcriptional condensates based on IDR-mCh-sspB images. GFP signals indirectly show the location of BioID-24×SunTag. Streptavidin-Alexa647 shows the biotinylated proteins in cells. For ‘All’, GFP accumulates in the transcriptional condensates and Streptavidin-Alexa647 also accumulates in the condensates. It suggests that biotinylation occurs inside the condensates by BioID-24×SunTag. However, the other three controls do not show Streptavidin-Alexa647 accumulations in transcriptional condensates. For ‘Cont1’, GFP does not accumulate into condensates, indicating that BioID-24×SunTag also does not accumulate. For ‘Cont2’, GFP accumulates in the condensates as ‘All’ experiment shows, but BioID does not efficiently biotinylate adjacent proteins due to a lack of biotin. For ‘Cont3’, GFP accumulates weakly in the condensates, but this accumulation is from a direct binding of sspB and LID, not from an enhancement of GFP from SunTag. Thus, it shows that the cells in this condition do not ex-press enough BioID-SunTag complexes, resulting in no biotinylated proteins in the condensates. Note that the background biotinylation levels in nucleus are also different. For ‘All’ and ‘Cont1’, biotinylation occurs mainly inside of nucleus, thus one can see the shape of nucleus and nucleoli, but for ‘Cont2’ and ‘Cont3’, biotinylation does not mainly occur in nucleus so that the signal level in nucleus is relatively lower than the signal level in cytoplasms. Statistical analysis of GFP relative intensity and Streptavidin-Alexa647 relative intensity are shown in FIG. 10I and J (Again, relative intensity means the peak intensity in the condensate site divided by the average intensity in background). For GFP, ‘All’ and ‘Cont2’ show accumulations into condensates. ‘Cont3’ shows little accumulations of GFP due to the direct interaction of sspB and LID, but ‘Cont1’ does not show any accumulations. Mean (+ mark in the box plot) and median (middle line in the box plot) values are 4.730 (±1.914, SD, n=36) and 4.169 for ‘All’, 1.118 (±0.069, SD, n=23) and 1.116 for ‘Cont1’, 4.548 (±2.455, SD, n=22) and 3.536 for ‘Cont2’, and 1.959 (±0.911, SD, n=21) and 1.724 for ‘Cont3’. For Streptavidin-Alexa647, only ‘All’ condition shows accumulations into condensates. Mean (+ mark in the box plot) and median (middle line in the box plot) values are 2.291 (±0.543, SD, n=36) and 2.245 for ‘All’, 1.116 (±0.049, SD, n=23) and 1.102 for ‘Cont1’, 1.181 (±0.107, SD, n=22) and 1.155 for ‘Cont2’, and 1.107 (±0.082, SD, n=21) and 1.117 for ‘Cont3’.

This proof-of-concept experiment clearly shows that BioID is accumulated into transcriptional condensates upon blue light and it biotinylates the adjacent proteins in the condensates. Thus, one concludes that the LiTEC system can drag BioID into condensates using blue light, and the dragged BioID successfully biotinylates proteins inside of the condensates.

### Mass Spectrometry

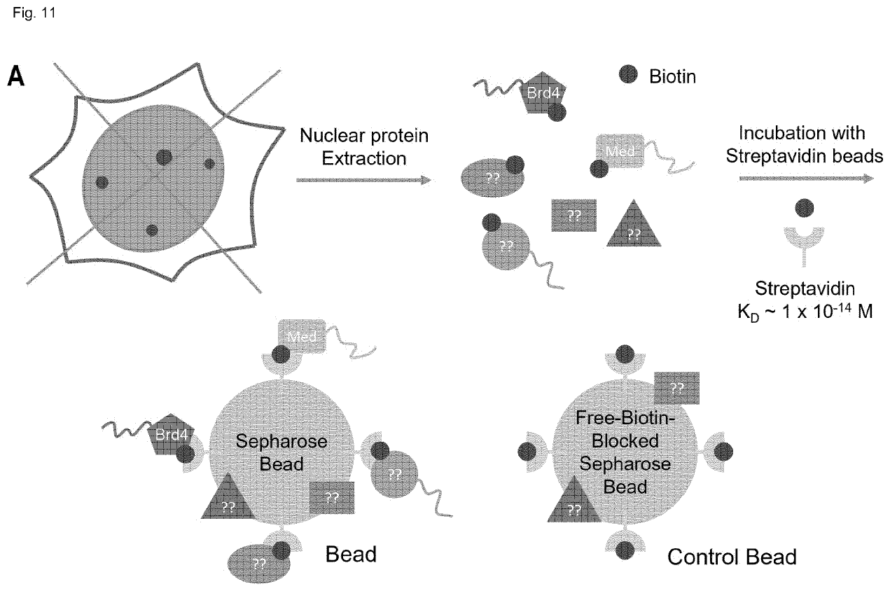

The number of cells is scaled up to prepare enough nuclear extracts for mass spectrometry experiments. The basic steps for mass spec sample preparation are shown in FIG. 11. First, cells grow under proper treatments such as blue light exposure, exogenous biotin incubation, and doxycycline incubation. For the first preliminary data, the conditions used in FIG. 10 are maintained. Then, cells are sacrificed and only nuclear extracts are isolated. The nuclear extracts contain biotinylated and non-biotinylated proteins. Since streptavidin has a very specific and extremely strong affinity to biotin (The dissociation constant is ~1×10⁻¹⁴ M), one can use streptavidin beads to pull down the biotinylated proteins. Note that, however, there are non-specific binding proteins to the bead so we also have to consider these proteins. ‘Control’ beads which are incubated with free biotin to block all available streptavidin. After blocking, the control beads are incubated with the nuclear extracts to check non-specific binding proteins. After reading out the mass spec results, one can then get a list of true biotinylated proteins by comparing lists from control beads and original beads. After the nuclear extract incubation with two types of streptavidin beads, the streptavidin-biotin bonding is broken by chemical processes, then desalted. In the end, the samples are loaded for LC-MS/MS analysis.

Similar to the imaging experiments in FIG. 10, four mass spec samples were obtained for four different conditions. The experiment's objective is to identify proteins in the small condensates, meaning that the number of proteins in the small condensates may be very small. Thus, the analysis focusses on highly biotinylated proteins rather than their abundance.

A pilot mass spectrometry experiment was performed with different conditions. For group ‘All’, blue light (BL) is on for 30 min, 50 μM Biotin (Bio) is incubated for 30 min (with BL), and 2 μg/ml Doxycycline is incubated for 48 hours. For control experiments, one of these treatments is removed.

Positive cofactor 4 (PC4), Cyclin-T1 (CCTN1), Cyclin-T2 (CCTN2), Cyclin-dependent kinase 9 (CDK9), Bromodomain-containing protein 4 (BRD4), and Mediator subunit 15 (MED15) were found to be highly enriched proteins and are regarded as proteins in transcriptional condensates. RNA polymerase II is not detected and CDK7 is also not detected in this experiment.

PC4, BRD4, MED15, CDK9, CCNT1, CCNT2 were identified as present and successfully labelled by a pilot MS analysis experiment. Their enrichment is over 2, indicating that they might be found in transcriptional condensates. Also, these proteins are functionally related to transcriptional activity. Especially, it is already known that transcriptional condensates contain BRD4 and Mediator, validating the result. CDK9, Cyclin-T1, and Cyclin-T2 are known as subunits of P-TEFb (Positive transcription elongation factor b), which helps to release the paused RNA pol II by phosphorylating CTD of Pol II Rpb1 subunit, DSIF, and NELF. It may indicate that transcriptional condensates might upregulate transcription by concentrating subunits of P-TEFb in condensates. Interestingly, RNA Polymerase II subunits are not found in all the results. Also, CDK7, which is related to the initiation of transcription, is not also found in the results.

### Validation of Mass Spec Result by Immunofluorescence

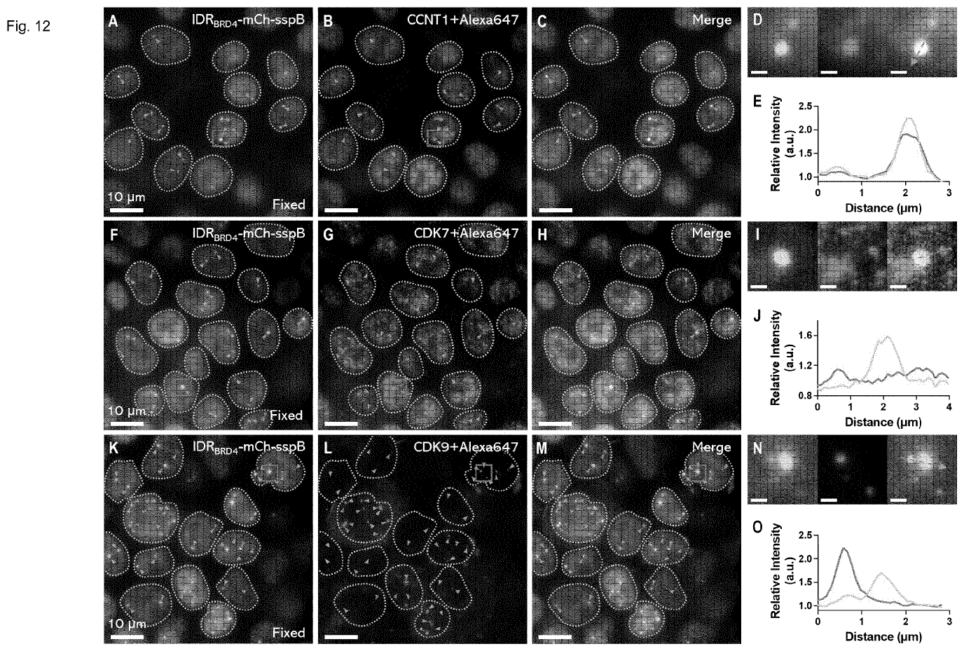

For the first experiment, anti-CCNT1, anti-CDK7, and anti-CDK9 were selected. CDK7 is not in the protein list from mass spectrometry results, but the inventors were curious how the different distribution CDK7 will show compared to the distribution of CCNT1 or CDK9 in nucleus, as the role of CDK7 is important to transcriptional activity. Also, it could be used for negative control. FIG. 12 shows the results of immunofluorescence for CCNT1, CDK7, and CDK9 in fixed cells. IDR-mCh-sspB (green) is used to find transcriptional condensates in nucleus (FIG. 12A, F, and K). Indirect immunofluorescence is adopted to visualize CCNT1, CDK7, and CKD9. A secondary antibody with Alexa647 is imaged and colored as magenta (FIG. 12B, G, and L). Then, the IDR image and the anti-protein image are overlaid to check their distribution and colocalization in nucleus (FIG. 12C, H, and M). A yellow dashed line shows the nucleus membrane of a cell. Plus, a red box in each IF is magnified and the relative intensity profile along a sky arrow line is shown in panel D-E, I-J, and N-O. There are orange arrows and sky-blue arrows. Both arrows indicate the location of transcriptional condensates based on the IDR images, but the sky-blue color is used if an IDR cluster is not colocalized with IF foci. The orange arrow indicates clusters colocalized with IF foci.

Similar to transcriptional condensates by IDR (FIG. 12A), CCNT1 forms foci with the uniform background level in nucleus (FIG. 12B). Most transcriptional condensates are colocalized with CCNT1 foci, but there are some condensates that do not contain CCNT1. In this case, it seems that big and strong transcriptional condensates mostly have CCNT1 inside of them (FIG. 12C). The relative intensity profiles in panel E confirm again that transcriptional condensates and CCNT1 clusters are colocalized. This result again indicates that BioID with LiTEC system detects CCNT1 successfully.

However, not like CCNT1, CDK7 are not colocalized with transcriptional condensates even though CDK7 forms a lot of foci in nucleus (FIG. 12G). From magnified images and the relative intensity profiles in panel I and J, it even seems that CDK7 is excluded for big and strong transcriptional condensates. It may mean that a transcriptional condensate is not related to transcription initiation. Also, it exactly shows the result of negative control, suggesting that the previous mass spectrometry result makes sense again.

CDK9 shows a lot of foci in nucleus (FIG. 12L). Interestingly, some condensates are colocalized with CDK9 foci but some are not (FIG. 12M). A region with red box is magnified as an example. Magnified images and the relative intensity profiles in panel N and O show that relatively small condensates are colocalized with small CDK9 foci but CDK9 does not show any foci at the location of big condensates. Generally, small transcriptional condensates with weak intensities are colocalized with small CDK9 foci but big transcriptional condensates with strong intensities are not colocalized with CDK9 foci. One can conclude that the size of transcriptional condensates may be correlated to their functionality, yet causality is not known. This result shows that BioID with LiTEC system is able to successfully detect CDK9, which even resides mostly in small transcriptional condensates.

In conclusion, immunofluorescence for CCNT1, CDK7, and CDK9 successfully validated the result of mass spectrometry. It seems that BRD4 IDR might be a basal scaffold for different types of transcriptional condensates which may have different functions in transcription.

### Methods

Standard protocols were followed (see Celis, Cell Biology: A Laboratory Handbook, Elsevier; Green and Sambrook, Molecular Cloning, CSH Press; Helgason and Miller, Basic Cell Culture Protocols, Springer)

### CITED REFERENCES

Forman-Kay and Mittag (2013), Structure 21, 1492-1499, 32-34
Guntas et al. (2015) PNAS 112, 112-117
Madan Babu, Biochem Soc Trans. (2016) Oct. 15; 44(5): 1185-1200
Uversky et al. Proteins: Struct., Funct., Bioinf. 41, 415-427
Trinkle-Mulcahy, F1000Research 2019, 8(F1000 Faculty Rev):135)
Zhou et al., Nature Biotechnology 40, 262-272 (2022);
Toettcher et al., Nature Methods 8, 837-839 (2011)
Crefcoeur et al., Nature Communications 4, Article: 1779 (2013)
Konermann et al. Nature 500, 472-476 (2013)
Yazawa et al., Nature Biotechnology 27, 941-945 (2009)
Kawano et al. Nature Communications 6, Article: 6256 (2015)
Strickland et al., Nature Methods 9, 379-384 (2012)
He et al., Nature Chemical Biology 17, 915-923 (2021)
Kaberniuk et al., Nature Methods 13, 591-597 (2016)
Redchuk et al., Nature Chemical Biology volume 13, 633-639 (2017)
Kuwasaki et al., Nature Biotechnology 40, 1672-1679 (2022)

All scientific publications and patent documents cited in the present specification are incorporated by reference herein.

Sequences:

SEQ ID NO 001: BRD4ΔN IDR:
MEEPVVAVSSPAVPPPTKVVAPPSSSDSSSDSSSDSDSSTDDSEEERAQRLAELQEQLKAVHEQLAALSQP
QQNKPKKKEKDKKEKKKEKHKRKEEVEENKKSKAKEPPPKKTKKNNSSNSNVSKKEPAPMKSKPPPTYESE
EEDKCKPMSYEEKRQLSLDINKLPGEKLGRVVHIIQSREPSLKNSNPDEIEIDFETLKPSTLRELERYVTS
CLRKKRKPQAEKVDVIAGSSKMKGFSSSESESSSESSSSDSEDSETEMAPKSKKKGHPGREQKKHHHHHHQ
QMQQAPAPVPQQPPPPPQQPPPPPPPQQQQQPPPPPPPPSMPQQAAPAMKSSPPPFIATQVPVLEPQLPGS
VFDPIGHFTQPILHLPQPELPPHLPQPPEHSTPPHLNQHAVVSPPALHNALPQQPSRPSNRAAALPPKPAR
PPAVSPALTQTPLLPQPPMAQPPQVLLEDEEPPAPPLTSMQMQLYLQQLQKVQPPTPLLPSVKVQSQPPPP
LPPPPHPSVQQQLQQQPPPPPPPQPQPPPQQQHQPPPRPVHLQPMQFSTHIQQPPPPQGQQPPHPPPGQQP
PPPQPAKPQQVIQHHHSPRHHKSDPYSTGHLREAPSPLMIHSPQMSQFQSLTHQSPPQQNVQPKKQELRAA
SVVQPQPLVVVKEEKIHSPIIRSEPFSPSLRPEPPKHPESIKAPVHLPQRPEMKPVDVGRPVIRPPEQNAP
PPGAPDKDKQKQEPKTPVAPKKDLKIKNMGSWASLVQKHPTTPSSTAKSSSDSFEQFRRAAREKEEREKAL
KAQAEHAEKEKERLRQERMRSREDEDALEQARRAHEEARRRQEQQQQQRQEQQQQQQQQAAAVAAAATPQA
QSSQPQSMLDQQRELARKREQERRRREAMAATIDMNFQSDLLSIFEENLF
SEQ ID NO 002: NELFA IDR:
MASMRESDTGLWLHNKLGATDELWAPPSIASLLTAAVIDNIRLCFHRLSSAVKLKLLLGTLHLPRRTVDEM
KAALMDIIQLATLDSDPWVLMVADILKSFPDTGSLNLDLEEQNPNVQDILGELREKVSECEASAMLPLECQ
YLNKNALTTLAGPLTPPVKHFQLKRKPKSATLRAELLQKSTETAQQLKRSAGVPFHAKGRGLLRKMDTTTP
LKGIPKQAPFRSPTTPSVFSPSGNRTPIPPSRTPLQKERGVKLLDISELNTVGAGREAKRRRKTLDTEVVE
KPTKEETVVENATPDYAAGLVSTQKLGSLNSEPTLPSTSYLPSTPSVVPASSYIPSSETPPAPPSREASRP
PEEPSAPSPTLPTQFKQRAPMYNSGLSPATPAPAAPTSPLTPTTPPAVTPTAQTPPVAMVAPQTQAPAPVQ
QQPKKNLSLTREQMFAAQEMFKTANKVTRPEKALILGFMAGSRENPCPEQGDVIQIKLSEHTEDLPKADGQ
GSTTMLVDTVFEMNYATGQWTRFKKYKPMTNVS
SEQ ID NO 003: Ssp3
SSPKRPKLLREYYDWLVDNSFTPYLVVDATYLGVNVPVEYVKDGQIVLNLSASATGNLQLTNDFIQFNARF
KGVSRELYIPMGAALAIYARENGDGVMFEPEEIYDELNIG
SEQ ID NO 004: iLID
LATTLERIEKNFVITDPRLPDNPIIFASDSFLQLTEYSREEILGRNCRFLQGPETDRATVRKIRDAIDNQT
EVTVQLINYTKSGKKFWNVFHLQPMRDYKGDVQYFIGVQLDGTERLHGAAEREAVCLIKKTAFQIAEAAND
ENYFG
SEQ ID NO 005: SunTag epitope:
EELLSKNYHLENEVARLKK
SEQ ID NO 006: SunTag full sequence GCN4_v4 (Tanenbaum et al., (2014)
Cell, 159(3):635-46):
DGIGSGGGGSGGEELLSKNYHLENEVARLKKGSGSGEELLSKNYHLENEVARLKKGSGSGEELLSKNYHLE
NEVARLKKGSGSGEELLSKNYHLENEVARLKKGSGSGEELLSKNYHLENEVARLKKGSGSGEELLSKNYHL
ENEVARLKKGSGSGEELLSKNYHLENEVARLKKGSGSGEELLSKNYHLENEVARLKKGSGSGEELLSKNYH
LENEVARLKKGSGSGEELLSKNYHLENEVARLKKGSGSGEELLSKNYHLENEVARLKKGSGSGEELLSKNY
HLENEVARLKKGSGSGEELLSKNYHLENEVARLKKGSGSGEELLSKNYHLENEVARLKKGSGSGEELLSKN
YHLENEVARLKKGSGSGEELLSKNYHLENEVARLKKGSGSGEELLSKNYHLENEVARLKKGSGSGEELLSK
NYHLENEVARLKKGSGSGEELLSKNYHLENEVARLKKGSGSGEELLSKNYHLENEVARLKKGSGSGEELLS
KNYHLENEVARLKKGSGSGEELLSKNYHLENEVARLKKGSGSGEELLSKDYHLENEVARLKKGSGSGEELL
SKNYHLENEVARLKKGSGSGQRPQGGGG
SEQ ID NO 007: BioID
IPLLNAKQILGQLDGGSVAVLPVVDSTNQYLLDRIGELKSGDACIAEYQQAGRGSRGRKWFSPFGANLYLS
MFWRLKRGPAAIGLGPVIGIVMAEALRKLGADKVRVKWPNDLYLQDRKLAGILVELAGITGDAAQIVIGAG
INVAMRRVEESVVNQGWITLQEAGINLDRNTLAAMLIRELRAALELFEQEGLAPYLSRWEKLDNFINRPVK
LIIGDKEIFGISRGIDKQGALLLEQDGVIKPWMGGEISLRSAEK
SEQ ID NO 008: BRD4ΔN-mCh-sspB;
MEEPVVAVSSPAVPPPTKVVAPPSSSDSSSDSSSDSDSSTDDSEEERAQRLAELQEQLKAVHEQLAALSQP
QQNKPKKKEKDKKEKKKEKHKRKEEVEENKKSKAKEPPPKKTKKNNSSNSNVSKKEPAPMKSKPPPTYESE
EEDKCKPMSYEEKRQLSLDINKLPGEKLGRVVHIIQSREPSLKNSNPDEIEIDFETLKPSTLRELERYVTS
CLRKKRKPQAEKVDVIAGSSKMKGFSSSESESSSESSSSDSEDSETEMAPKSKKKGHPGREQKKHHHHHHQ
QMQQAPAPVPQQPPPPPQQPPPPPPPQQQQQPPPPPPPPSMPQQAAPAMKSSPPPFIATQVPVLEPQLPGS
VFDPIGHFTQPILHLPQPELPPHLPQPPEHSTPPHLNQHAVVSPPALHNALPQQPSRPSNRAAALPPKPAR
PPAVSPALTQTPLLPQPPMAQPPQVLLEDEEPPAPPLTSMQMQLYLQQLQKVQPPTPLLPSVKVQSQPPPP
LPPPPHPSVQQQLQQQPPPPPPPQPQPPPQQQHQPPPRPVHLQPMQFSTHIQQPPPPQGQQPPHPPPGQQP
PPPQPAKPQQVIQHHHSPRHHKSDPYSTGHLREAPSPLMIHSPQMSQFQSLTHQSPPQQNVQPKKQELRAA
SVVQPQPLVVVKEEKIHSPIIRSEPFSPSLRPEPPKHPESIKAPVHLPQRPEMKPVDVGRPVIRPPEQNAP
PPGAPDKDKQKQEPKTPVAPKKDLKIKNMGSWASLVQKHPTTPSSTAKSSSDSFEQFRRAAREKEEREKAL
KAQAEHAEKEKERLRQERMRSREDEDALEQARRAHEEARRRQEQQQQQRQEQQQQQQQQAAAVAAAATPQA
QSSQPQSMLDQQRELARKREQERRRREAMAATIDMNFQSDLLSIFEENLFGGGMVSKGEEDNMAIIKEFMR
FKVHMEGSVNGHEFEIEGEGEGRPYEGTQTAKLKVTKGGPLPFAWDILSPQFMYGSKAYVKHPADIPDYLK
LSFPEGFKWERVMNFEDGGVVTVTQDSSLQDGEFIYKVKLRGTNFPSDGPVMQKKTMGWEASSERMYPEDG
ALKGEIKQRLKLKDGGHYDAEVKTTYKAKKPVQLPGAYNVNIKLDITSHNEDYTIVEQYERAEGRHSTGGM
DELYKAAGSGGTSGGSSPKRPKLLREYYDWLVDNSFTPYLVVDATYLGVNVPVEYVKDGQIVLNLSASATG
NLQLTNDFIQFNARFKGVSRELYIPMGAALAIYARENGDGVMFEPEEIYDELNIG
Underlined: Human BRD4 462aa-1362aa, Uniprot ID Q60885
Italic: mCherry
Underlined italic : sspB Nano (Guntas et al., (2014) PNAS, 112 (1) 112-117)
Normal font: amino acid linker
SEQ ID NO 009: NELFA-mCh-sspB
MASMRESDTGLWLHNKLGATDELWAPPSIASLLTAAVIDNIRLCFHRLSSAVKLKLLLGTLHLPRRTVDEM
KAALMDIIQLATLDSDPWVLMVADILKSFPDTGSLNLDLEEQNPNVQDILGELREKVSECEASAMLPLECQ
YLNKNALTTLAGPLTPPVKHFQLKRKPKSATLRAELLQKSTETAQQLKRSAGVPFHAKGRGLLRKMDTTTP
LKGIPKQAPFRSPTTPSVFSPSGNRTPIPPSRTPLQKERGVKLLDISELNTVGAGREAKRRRKTLDTEVVE
KPTKEETVVENATPDYAAGLVSTQKLGSLNSEPTLPSTSYLPSTPSVVPASSYIPSSETPPAPPSREASRP
PEEPSAPSPTLPTQFKQRAPMYNSGLSPATPAPAAPTSPLTPTTPPAVTPTAQTPPVAMVAPQTQAPAPVQ
QQPKKNLSLTREQMFAAQEMFKTANKVTRPEKALILGFMAGSRENPCPEQGDVIQIKLSEHTEDLPKADGQ
GSTTMLVDTVFEMNYATGQWTRFKKYKPMTNVSGGGMVSKGEEDNMAIIKEFMRFKVHMEGSVNGHEFEIE
GEGEGRPYEGTQTAKLKVTKGGPLPFAWDILSPQFMYGSKAYVKHPADIPDYLKLSFPEGFKWERVMNFED
GGVVTVTQDSSLQDGEFIYKVKLRGTNFPSDGPVMQKKTMGWEASSERMYPEDGALKGEIKQRLKLKDGGH
YDAEVKTTYKAKKPVQLPGAYNVNIKLDITSHNEDYTIVEQYERAEGRHSTGGMDELYKAAGSGGTSGGSS
PKRPKLLREYYDWLVDNSFTPYLVVDATYLGVNVPVEYVKDGQIVLNLSASATGNLQLTNDFIQFNARFKG
VSRELYIPMGAALAIYARENGDGVMFEPEEIYDELNIG
Underlined: mouse NELFA, Uniprot ID Q8BG30
Italic : mCherry
Underlined italic: sspB Nano (Guntas et al., (2014) PNAS, 112 (1) 112-117)
Normal font: acid linker
SEQ ID NO 010: BRD4ΔN-sspB;
MEEPVVAVSSPAVPPPTKVVAPPSSSDSSSDSSSDSDSSTDDSEEERAQRLAELQEQLKAVHEQLAALSQP
QQNKPKKKEKDKKEKKKEKHKRKEEVEENKKSKAKEPPPKKTKKNNSSNSNVSKKEPAPMKSKPPPTYESE
EEDKCKPMSYEEKRQLSLDINKLPGEKLGRVVHIIQSREPSLKNSNPDEIEIDFETLKPSTLRELERYVTS
CLRKKRKPQAEKVDVIAGSSKMKGFSSSESESSSESSSSDSEDSETEMAPKSKKKGHPGREQKKHHHHHHQ
QMQQAPAPVPQQPPPPPQQPPPPPPPQQQQQPPPPPPPPSMPQQAAPAMKSSPPPFIATQVPVLEPQLPGS
VFDPIGHFTQPILHLPQPELPPHLPQPPEHSTPPHLNQHAVVSPPALHNALPQQPSRPSNRAAALPPKPAR
PPAVSPALTQTPLLPQPPMAQPPQVLLEDEEPPAPPLTSMQMQLYLQQLQKVQPPTPLLPSVKVQSQPPPP
LPPPPHPSVQQQLQQQPPPPPPPQPQPPPQQQHQPPPRPVHLQPMQFSTHIQQPPPPQGQQPPHPPPGQQP
PPPQPAKPQQVIQHHHSPRHHKSDPYSTGHLREAPSPLMIHSPQMSQFQSLTHQSPPQQNVQPKKQELRAA
SVVQPQPLVVVKEEKIHSPIIRSEPFSPSLRPEPPKHPESIKAPVHLPQRPEMKPVDVGRPVIRPPEQNAP
PPGAPDKDKQKQEPKTPVAPKKDLKIKNMGSWASLVQKHPTTPSSTAKSSSDSFEQFRRAAREKEEREKAL
KAQAEHAEKEKERLRQERMRSREDEDALEQARRAHEEARRRQEQQQQQRQEQQQQQQQQAAAVAAAATPQA
QSSQPQSMLDQQRELARKREQERRRREAMAATIDMNFQSDLLSIFEENLFAAGSGGTSGGSSPKRPKLLRE
YYDWLVDNSFTPYLVVDATYLGVNVPVEYVKDGQIVLNLSASATGNLQLTNDFIQFNARFKGVSRELYIPM
GAALAIYARENGDGVMFEPEEIYDELNIG
SEQ ID NO 011: NELFA-sspB
MASMRESDTGLWLHNKLGATDELWAPPSIASLLTAAVIDNIRLCFHRLSSAVKLKLLLGTLHLPRRTVDEM
KAALMDIIQLATLDSDPWVLMVADILKSFPDTGSLNLDLEEQNPNVQDILGELREKVSECEASAMLPLECQ
YLNKNALTTLAGPLTPPVKHFQLKRKPKSATLRAELLQKSTETAQQLKRSAGVPFHAKGRGLLRKMDTTTP
LKGIPKQAPFRSPTTPSVFSPSGNRTPIPPSRTPLQKERGVKLLDISELNTVGAGREAKRRRKTLDTEVVE
KPTKEETVVENATPDYAAGLVSTQKLGSLNSEPTLPSTSYLPSTPSVVPASSYIPSSETPPAPPSREASRP
PEEPSAPSPTLPTQFKQRAPMYNSGLSPATPAPAAPTSPLTPTTPPAVTPTAQTPPVAMVAPQTQAPAPVQ
QQPKKNLSLTREQMFAAQEMFKTANKVTRPEKALILGFMAGSRENPCPEQGDVIQIKLSEHTEDLPKADGQ
GSTTMLVDTVFEMNYATGQWTRFKKYKPMTNVSAAGSGGTSGGSSPKRPKLLREYYDWLVDNSFTPYLVVD
ATYLGVNVPVEYVKDGQIVLNLSASATGNLQLTNDFIQFNARFKGVSRELYIPMGAALAIYARENGDGVMF
EPEEIYDELNIG
SEQ ID NO 012: scFv-sfGFP-GB1-iLID-NLS
MGPDIVMTQSPSSLSASVGDRVTITCRSSTGAVTTSNYASWVQEKPGKLFKGLIGGINNRAPGVPSRFSGS
LIGDKATLTISSLQPEDFATYFCALWYSNHWVFGQGTKVELKRGGGGSGGGGSGGGGSSGGGSEVKLLESG
GGLVQPGGSLKLSCAVSGFSLTDYGVNWVRQAPGRGLEWIGVIWGDGITDYNSALKDRFIISKDNGKNTVY
LQMSKVRSDDTALYYCVTGLFDYWGQGTLVTVSSYPYDVPDYAGGGGGSGGGGSGGGGSGGGGSLDPGGGG
SGSKGEELFTGVVPILVELDGDVNGHKFSVRGEGEGDATNGKLTLKFICTTGKLPVPWPTLVTTLTYGVQC
FSRYPDHMKRHDFFKSAMPEGYVQERTISFKDDGTYKTRAEVKFEGDTLVNRIELKGIDFKEDGNILGHKL
EYNFNSHNVYITADKQKNGIKANFKIRHNVEDGSVQLADHYQQNTPIGDGPVLLPDNHYLSTQSVLSKDPN
EKRDHMVLLEFVTAAGITHGMDELYKGGGRTEEYKLILNGKTLKGETTTEAVDAATAEKVFKQYANDNGVD
GEWTYDDATKTFTVTEGGGSGGGTRLATTLERIEKNFVITDPRLPDNPIIFASDSFLQLTEYSREEILGRN
CRFLQGPETDRATVRKIRDAIDNQTEVTVQLINYTKSGKKFWNVFHLQPMRDYKGDVQYFIGVQLDGTERL
HGAAEREAVCLIKKTAFQIAEAANDENYFGGGGSGGGTSPKKKRKV
Italic: single chain variable fragment (scFv)
bold: super-foldable GFP
Grey: GB1 (B1 domain of Streptococcal protein G)
Bold italic: improved Light-inducible dimer
normal: nuclear localization signal (NLS)
underlined : amino acid linker
SEQ ID NO 013: BioID-HA-2xNLS-24xSunTag
MEFIPLLNAKQILGQLDGGSVAVLPVVDSTNQYLLDRIGELKSGDACIAEYQQAGRGSRGRKWFSPFGANL
YLSMFWRLKRGPAAIGLGPVIGIVMAEALRKLGADKVRVKWPNDLYLQDRKLAGILVELAGITGDAAQIVI
GAGINVAMRRVEESVVNQGWITLQEAGINLDRNTLAAMLIRELRAALELFEQEGLAPYLSRWEKLDNFINR
PVKLIIGDKEIFGISRGIDKQGALLLEQDGVIKPWMGGEISLRSAEKLDPGGGGSGYPYDVPDYASLGSGS
PKKKRKVEDPKKKRKVDGIGSGGGGSGGEELLSKNYHLENEVARLKKGSGSGEELLSKNYHLENEVARLKK
GSGSGEELLSKNYHLENEVARLKKGSGSGEELLSKNYHLENEVARLKKGSGSGEELLSKNYHLENEVARLK
KGSGSGEELLSKNYHLENEVARLKKGSGSGEELLSKNYHLENEVARLKKGSGSGEELLSKNYHLENEVARL
KKGSGSGEELLSKNYHLENEVARLKKGSGSGEELLSKNYHLENEVARLKKGSGSGEELLSKNYHLENEVAR
LKKGSGSGEELLSKNYHLENEVARLKKGSGSGEELLSKNYHLENEVARLKKGSGSGEELLSKNYHLENEVA
RLKKGSGSGEELLSKNYHLENEVARLKKGSGSGEELLSKNYHLENEVARLKKGSGSGEELLSKNYHLENEV
ARLKKGSGSGEELLSKNYHLENEVARLKKGSGSGEELLSKNYHLENEVARLKKGSGSGEELLSKNYHLENE
VARLKKGSGSGEELLSKNYHLENEVARLKKGSGSGEELLSKNYHLENEVARLKKGSGSGEELLSKDYHLEN
EVARLKKGSGSGEELLSKNYHLENEVARLKKGSGSGQRPQGGGGPKKKRKV
Italic: miniTurboID (Branon et al., (2018) Nat. Biotechnol., 36(9):880-887)
Italic bold : HA tag
normal: SunTag, GCN4_v4 (Tanenbaum et al., (2014) Cell, 159(3):635-46)
bold : nuclear localization signal (NLS)
Underlined: amino acid linker

1. A system for covalently modifying a target,
the target being associated to an endogenous intracellular condensate formed by an intracellular protein comprising an Intrinsically Disordered Region (IDR),
or a system for modifying a chemical or physical property of an endogenous intracellular condensate,
said system comprising:
a. an IDR sequence tract comprising said Intrinsically Disordered Region and
b. an effector domain capable of modifying said target;
wherein the IDR sequence tract and the effector domain are located on distinct polypeptide molecules that can be induced to associate in response to light.

2. The system according to claim 1, wherein the protein comprising an IDR is a protein located in the nucleus of a eukaryote cell.

3. The system according to claim 1, wherein the protein comprising an IDR is selected from the group comprised of the proteins: BRD4, NELFA, NELFB, CDK9, P-TEFb, Mediator complex; RNA Polymerase (RPB1);
particularly wherein the protein comprising an IDR is BRD4 or NELFA.

4. The system according to claim 1, wherein the IDR sequence tract is selected from SEQ ID NO 001 and SEQ ID NO 002 (ΔN-BDR4 IDR; NELFA IDR), or a sequence variant thereof characterized by at least 85% sequence identity to any of SEQ ID NO 001 and SEQ ID NO 002.

5. The system according to claim 1, wherein the target is a protein, particularly an intracellular protein, more particularly an intracellular nuclear protein, even more particularly a nuclear protein associated to RNA polymerase II activity.

6. The system according to claim 1, wherein the effector polypeptide capable of covalently modifying said target is selected from a polypeptide capable of
a. biotinylating,
b. ubiquitinylating,
c. methylating,
d. demethylating,
e. acetylating,
f. deacetylating,
g. phosphorylating,
h. dephosphorylating
a target.

7. The system according to claim 1, wherein association of a first fusion polypeptide comprising
the IDR tract and another fusion peptide comprising the effector domain is facilitated by a light-induced binding partner pair,
the light-induced binding partner pair consisting of a first binding partner and a second binding partner, wherein the first and second binding partners associate in presence of light,
and wherein one of the binding partners is part of the first fusion polypeptide and the other of the binding partners is associated with the effector domain.

8. The system according to claim 7, wherein the light induced binding partner pair is SspB and iLID (SEQ ID NO 003 and 004).

9. The system according to claim 1, wherein
the system consists of:
a. a first fusion polypeptide comprising an IDR sequence tract comprising said Intrinsically Disordered Region, [an optional first fluorescence marker polypeptide] and a first component of a light-induced binding partner pair;
b. a second fusion polypeptide comprising a second component of said light-induced binding partner pair, [an optional second fluorescence marker polypeptide] and a binding domain capable of specifically binding to a non-endogenous peptide epitope;
c. a third fusion polypeptide comprising a plurality of said non-endogenous peptide epitopes and an effector domain capable of covalently modifying said target.

10. The system according to claim 9, wherein said binding domain capable of specifically binding to a non-endogenous peptide epitope is an scFv (single chain variable) antibody fragment.

11. A nucleic acid sequence, or a plurality of nucleic acid sequences, encoding the system as specified in claim 1.

12. A method for modifying a target (particularly a target protein) associated to an endogenous intracellular condensate formed by an intracellular protein comprising an Intrinsically Disordered Region, said method comprising the steps:
a) providing a cell comprising an intracellular protein comprising an Intrinsically Disordered Region;
b) expressing in said cell a system as specified in claim 1.

13. The method according to claim 12, wherein the method further comprises the steps of
c) collecting a preparation comprising target biomolecules (particularly proteins) from said cell,
d) isolating target biomolecules (particularly proteins) modified by said effector polypeptide capable of modifying the target biomolecules.

14. The method according to claim 12, wherein the effector polypeptide is capable of biotinylating target biomolecules, and isolating biotinylated target biomolecules is attained by binding biotinylated proteins from the preparation comprising biomolecules to a matrix.


SEQUENCE LISTING (13 sequences)

SEQ ID NO: 1 - protein, 902 aa, synthetic construct
MEEPVVAVSSPAVPPPTKVVAPPSSSDSSSDSSSDSDSSTDDSEEERAQRLAELQEQLKAVHEQLAALSQPQQNKPKKKEKDKKEKKKEKHKRKEEVEENKKSKAKEPPPKKTKKNNSSNSNVSKKEPAPMKSKPPPTYESEEEDKCKPMSYEEKRQLSLDINKLPGEKLGRVVHIIQSREPSLKNSNPDEIEIDFETLKPSTLRELERYVTSCLRKKRKPQAEKVDVIAGSSKMKGFSSSESESSSESSSSDSEDSETEMAPKSKKKGHPGREQKKHHHHHHQQMQQAPAPVPQQPPPPPQQPPPPPPPQQQQQPPPPPPPPSMPQQAAPAMKSSPPPFIATQVPVLEPQLPGSVFDPIGHFTQPILHLPQPELPPHLPQPPEHSTPPHLNQHAVVSPPALHNALPQQPSRPSNRAAALPPKPARPPAVSPALTQTPLLPQPPMAQPPQVLLEDEEPPAPPLTSMQMQLYLQQLQKVQPPTPLLPSVKVQSQPPPPLPPPPHPSVQQQLQQQPPPPPPPQPQPPPQQQHQPPPRPVHLQPMQFSTHIQQPPPPQGQQPPHPPPGQQPPPPQPAKPQQVIQHHHSPRHHKSDPYSTGHLREAPSPLMIHSPQMSQFQSLTHQSPPQQNVQPKKQELRAASVVQPQPLVVVKEEKIHSPIIRSEPFSPSLRPEPPKHPESIKAPVHLPQRPEMKPVDVGRPVIRPPEQNAPPPGAPDKDKQKQEPKTPVAPKKDLKIKNMGSWASLVQKHPTTPSSTAKSSSDSFEQFRRAAREKEEREKALKAQAEHAEKEKERLRQERMRSREDEDALEQARRAHEEARRRQEQQQQQRQEQQQQQQQQAAAVAAAATPQAQSSQPQSMLDQQRELARKREQERRRREAMAATIDMNFQSDLLSIFEENLF

SEQ ID NO: 2 - protein, 530 aa, synthetic construct
MASMRESDTGLWLHNKLGATDELWAPPSIASLLTAAVIDNIRLCFHRLSSAVKLKLLLGTLHLPRRTVDEMKAALMDIIQLATLDSDPWVLMVADILKSFPDTGSLNLDLEEQNPNVQDILGELREKVSECEASAMLPLECQYLNKNALTTLAGPLTPPVKHFQLKRKPKSATLRAELLQKSTETAQQLKRSAGVPFHAKGRGLLRKMDTTTPLKGIPKQAPFRSPTTPSVFSPSGNRTPIPPSRTPLQKERGVKLLDISELNTVGAGREAKRRRKTLDTEVVEKPTKEETVVENATPDYAAGLVSTQKLGSLNSEPTLPSTSYLPSTPSVVPASSYIPSSETPPAPPSREASRPPEEPSAPSPTLPTQFKQRAPMYNSGLSPATPAPAAPTSPLTPTTPPAVTPTAQTPPVAMVAPQTQAPAPVQQQPKKNLSLTREQMFAAQEMFKTANKVTRPEKALILGFMAGSRENPCPEQGDVIQIKLSEHTEDLPKADGQGSTTMLVDTVFEMNYATGQWTRFKKYKPMTNVS

SEQ ID NO: 3 - protein, 111 aa, synthetic construct
SSPKRPKLLREYYDWLVDNSFTPYLVVDATYLGVNVPVEYVKDGQIVLNLSASATGNLQLTNDFIQFNARFKGVSRELYIPMGAALAIYARENGDGVMFEPEEIYDELNIG

SEQ ID NO: 4 - protein, 147 aa, synthetic construct
LATTLERIEKNFVITDPRLPDNPIIFASDSFLQLTEYSREEILGRNCRFLQGPETDRATVRKIRDAIDNQTEVTVQLINYTKSGKKFWNVFHLQPMRDYKGDVQYFIGVQLDGTERLHGAAEREAVCLIKKTAFQIAEAANDENYFG

SEQ ID NO: 5 - protein, 19 aa, synthetic construct
EELLSKNYHLENEVARLKK

SEQ ID NO: 6 - protein, 596 aa, synthetic construct
DGIGSGGGGSGGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKDYHLENEVARLKKGSGSGEELLSKNYHLENEVARLKKGSGSGQRPQGGGG

SEQ ID NO: 7 - protein, 257 aa, synthetic construct
IPLLNAKQILGQLDGGSVAVLPVVDSTNQYLLDRIGELKSGDACIAEYQQAGRGSRGRKWFSPFGANLYLSMFWRLKRGPAAIGLGPVIGIVMAEALRKLGADKVRVKWPNDLYLQDRKLAGILVELAGITGDAAQIVIGAGINVAMRRVEESVVNQGWITLQEAGINLDRNTLAAMLIRELRAALELFEQEGLAPYLSRWEKLDNFINRPVKLIIGDKEIFGISRGIDKQGALLLEQDGVIKPWMGGEISLRSAEK

SEQ ID NO: 8 - protein, 1262 aa, synthetic construct
MEEPVVAVSSPAVPPPTKVVAPPSSSDSSSDSSSDSDSSTDDSEEERAQRLAELQEQLKAVHEQLAALSQPQQNKPKKKEKDKKEKKKEKHKRKEEVEENKKSKAKEPPPKKTKKNNSSNSNVSKKEPAPMKSKPPPTYESEEEDKCKPMSYEEKRQLSLDINKLPGEKLGRVVHIIQSREPSLKNSNPDEIEIDFETLKPSTLRELERYVTSCLRKKRKPQAEKVDVIAGSSKMKGFSSSESESSSESSSSDSEDSETEMAPKSKKKGHPGREQKKHHHHHHQQMQQAPAPVPQQPPPPPQQPPPPPPPQQQQQPPPPPPPPSMPQQAAPAMKSSPPPFIATQVPVLEPQLPGSVFDPIGHFTQPILHLPQPELPPHLPQPPEHSTPPHLNQHAVVSPPALHNALPQQPSRPSNRAAALPPKPARPPAVSPALTQTPLLPQPPMAQPPQVLLEDEEPPAPPLTSMQMQLYLQQLQKVQPPTPLLPSVKVQSQPPPPLPPPPHPSVQQQLQQQPPPPPPPQPQPPPQQQHQPPPRPVHLQPMQFSTHIQQPPPPQGQQPPHPPPGQQPPPPQPAKPQQVIQHHHSPRHHKSDPYSTGHLREAPSPLMIHSPQMSQFQSLTHQSPPQQNVQPKKQELRAASVVQPQPLVVVKEEKIHSPIIRSEPFSPSLRPEPPKHPESIKAPVHLPQRPEMKPVDVGRPVIRPPEQNAPPPGAPDKDKQKQEPKTPVAPKKDLKIKNMGSWASLVQKHPTTPSSTAKSSSDSFEQFRRAAREKEEREKALKAQAEHAEKEKERLRQERMRSREDEDALEQARRAHEEARRRQEQQQQQRQEQQQQQQQQAAAVAAAATPQAQSSQPQSMLDQQRELARKREQERRRREAMAATIDMNFQSDLLSIFEENLFGGGMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKAAGSGGTSGGSSPKRPKLLREYYDWLVDNSFTPYLVVDATYLGVNVPVEYVKDGQIVLNLSASATGNLQLTNDFIQFNARFKGVSRELYIPMGAALAIYARENGDGVMFEPEEIYDELNIG

SEQ ID NO: 9 - protein, 890 aa, synthetic construct
MASMRESDTGLWLHNKLGATDELWAPPSIASLLTAAVIDNIRLCFHRLSSAVKLKLLLGTLHLPRRTVDEMKAALMDIIQLATLDSDPWVLMVADILKSFPDTGSLNLDLEEQNPNVQDILGELREKVSECEASAMLPLECQYLNKNALTTLAGPLTPPVKHFQLKRKPKSATLRAELLQKSTETAQQLKRSAGVPFHAKGRGLLRKMDTTTPLKGIPKQAPFRSPTTPSVFSPSGNRTPIPPSRTPLQKERGVKLLDISELNTVGAGREAKRRRKTLDTEVVEKPTKEETVVENATPDYAAGLVSTQKLGSLNSEPTLPSTSYLPSTPSVVPASSYIPSSETPPAPPSREASRPPEEPSAPSPTLPTQFKQRAPMYNSGLSPATPAPAAPTSPLTPTTPPAVTPTAQTPPVAMVAPQTQAPAPVQQQPKKNLSLTREQMFAAQEMFKTANKVTRPEKALILGFMAGSRENPCPEQGDVIQIKLSEHTEDLPKADGQGSTTMLVDTVFEMNYATGQWTRFKKYKPMTNVSGGGMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKAAGSGGTSGGSSPKRPKLLREYYDWLVDNSFTPYLVVDATYLGVNVPVEYVKDGQIVLNLSASATGNLQLTNDFIQFNARFKGVSRELYIPMGAALAIYARENGDGVMFEPEEIYDELNIG

SEQ ID NO: 10 - protein, 1023 aa, synthetic construct
MEEPVVAVSSPAVPPPTKVVAPPSSSDSSSDSSSDSDSSTDDSEEERAQRLAELQEQLKAVHEQLAALSQPQQNKPKKKEKDKKEKKKEKHKRKEEVEENKKSKAKEPPPKKTKKNNSSNSNVSKKEPAPMKSKPPPTYESEEEDKCKPMSYEEKRQLSLDINKLPGEKLGRVVHIIQSREPSLKNSNPDEIEIDFETLKPSTLRELERYVTSCLRKKRKPQAEKVDVIAGSSKMKGFSSSESESSSESSSSDSEDSETEMAPKSKKKGHPGREQKKHHHHHHQQMQQAPAPVPQQPPPPPQQPPPPPPPQQQQQPPPPPPPPSMPQQAAPAMKSSPPPFIATQVPVLEPQLPGSVFDPIGHFTQPILHLPQPELPPHLPQPPEHSTPPHLNQHAVVSPPALHNALPQQPSRPSNRAAALPPKPARPPAVSPALTQTPLLPQPPMAQPPQVLLEDEEPPAPPLTSMQMQLYLQQLQKVQPPTPLLPSVKVQSQPPPPLPPPPHPSVQQQLQQQPPPPPPPQPQPPPQQQHQPPPRPVHLQPMQFSTHIQQPPPPQGQQPPHPPPGQQPPPPQPAKPQQVIQHHHSPRHHKSDPYSTGHLREAPSPLMIHSPQMSQFQSLTHQSPPQQNVQPKKQELRAASVVQPQPLVVVKEEKIHSPIIRSEPFSPSLRPEPPKHPESIKAPVHLPQRPEMKPVDVGRPVIRPPEQNAPPPGAPDKDKQKQEPKTPVAPKKDLKIKNMGSWASLVQKHPTTPSSTAKSSSDSFEQFRRAAREKEEREKALKAQAEHAEKEKERLRQERMRSREDEDALEQARRAHEEARRRQEQQQQQRQEQQQQQQQQAAAVAAAATPQAQSSQPQSMLDQQRELARKREQERRRREAMAATIDMNFQSDLLSIFEENLFAAGSGGTSGGSSPKRPKLLREYYDWLVDNSFTPYLVVDATYLGVNVPVEYVKDGQIVLNLSASATGNLQLTNDFIQFNARFKGVSRELYIPMGAALAIYARENGDGVMFEPEEIYDELNIG

SEQ ID NO: 11 - protein, 651 aa, synthetic construct
MASMRESDTGLWLHNKLGATDELWAPPSIASLLTAAVIDNIRLCFHRLSSAVKLKLLLGTLHLPRRTVDEMKAALMDIIQLATLDSDPWVLMVADILKSFPDTGSLNLDLEEQNPNVQDILGELREKVSECEASAMLPLECQYLNKNALTTLAGPLTPPVKHFQLKRKPKSATLRAELLQKSTETAQQLKRSAGVPFHAKGRGLLRKMDTTTPLKGIPKQAPFRSPTTPSVFSPSGNRTPIPPSRTPLQKERGVKLLDISELNTVGAGREAKRRRKTLDTEVVEKPTKEETVVENATPDYAAGLVSTQKLGSLNSEPTLPSTSYLPSTPSVVPASSYIPSSETPPAPPSREASRPPEEPSAPSPTLPTQFKQRAPMYNSGLSPATPAPAAPTSPLTPTTPPAVTPTAQTPPVAMVAPQTQAPAPVQQQPKKNLSLTREQMFAAQEMFKTANKVTRPEKALILGFMAGSRENPCPEQGDVIQIKLSEHTEDLPKADGQGSTTMLVDTVFEMNYATGQWTRFKKYKPMTNVSAAGSGGTSGGSSPKRPKLLREYYDWLVDNSFTPYLVVDATYLGVNVPVEYVKDGQIVLNLSASATGNLQLTNDFIQFNARFKGVSRELYIPMGAALAIYARENGDGVMFEPEEIYDELNIG

SEQ ID NO: 12 - protein, 756 aa, synthetic construct
MGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGSLDPGGGGSGSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGGRTEEYKLILNGKTLKGETTTEAVDAATAEKVFKQYANDNGVDGEWTYDDATKTFTVTEGGGSGGGTRLATTLERIEKNFVITDPRLPDNPIIFASDSFLQLTEYSREEILGRNCRFLQGPETDRATVRKIRDAIDNQTEVTVQLINYTKSGKKFWNVFHLQPMRDYKGDVQYFIGVQLDGTERLHGAAEREAVCLIKKTAFQIAEAANDENYFGGGGSGGGTSPKKKRKV

SEQ ID NO: 13 - protein, 903 aa, synthetic construct
MEFIPLLNAKQILGQLDGGSVAVLPVVDSTNQYLLDRIGELKSGDACIAEYQQAGRGSRGRKWFSPFGANLYLSMFWRLKRGPAAIGLGPVIGIVMAEALRKLGADKVRVKWPNDLYLQDRKLAGILVELAGITGDAAQIVIGAGINVAMRRVEESVVNQGWITLQEAGINLDRNTLAAMLIRELRAALELFEQEGLAPYLSRWEKLDNFINRPVKLIIGDKEIFGISRGIDKQGALLLEQDGVIKPWMGGEISLRSAEKLDPGGGGSGYPYDVPDYASLGSGSPKKKRKVEDPKKKRKVDGIGSGGGGSGGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKDYHLENEVARLKKGSGSGEELLSKNYHLENEVARLKKGSGSGQRPQGGGGPKKKRKV